SEQUENCE LISTING (82 sequences)

SEQ ID NO: 1 - protein, 92 aa, ARTIFICIAL SEQUENCE
  Synthetic Construct
PNIQNPDPAVYQLRDSKSSDKSVCLFTDFDSQTNVSQSKDSDVYITDKTVLDMRSMDFKSNSAVAWSNKSDFACANAFNNSIIPEDTFFPSP

SEQ ID NO: 2 - protein, 90 aa, ARTIFICIAL SEQUENCE
  Synthetic Construct
IQNPDPAVYQLRDSKSSDKSVCLFTDFDSQTNVSQSKDSDVYITDKTVLDMRSMDFKSNSAVAWSNKSDFACANAFNNSIIPEDTFFPSP

SEQ ID NO: 3 - protein, 97 aa, ARTIFICIAL SEQUENCE
  Synthetic Construct
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVK

SEQ ID NO: 4 - protein, 95 aa, ARTIFICIAL SEQUENCE
  Synthetic Construct
IQNPDPAVYQLRDSKSSDKSVCLFTDFDSQTNVSQSKDSDVYITDKTVLDMRSMDFKSNSAVAWSNKSDFACANAFNNSIIPEDTFFPSPEPKSC

SEQ ID NO: 5 - protein, 7 aa, ARTIFICIAL SEQUENCE
  Synthetic Construct
ESSCDVK

SEQ ID NO: 6 - protein, 5 aa, ARTIFICIAL SEQUENCE
  Synthetic Construct
EPKSC

SEQ ID NO: 7 - protein, 96 aa, ARTIFICIAL SEQUENCE
  Synthetic Construct
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDV

SEQ ID NO: 8 - protein, 99 aa, ARTIFICIAL SEQUENCE
  Synthetic Construct
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVGGG

SEQ ID NO: 9 - protein, 115 aa, ARTIFICIAL SEQUENCE
  Synthetic Construct
IQNPDPAVYQLRDSKSSDKSVCLFTDFDSQTNVSQSKDSDVYITDKTVLDMRSMDFKSNSAVAWSNKSDFACANAFNNSIIPEDTFFPSPGGGGSGGGGSGGGGSGGGGSGGGGS

SEQ ID NO: 10 - protein, 135 aa, ARTIFICIAL SEQUENCE
  Synthetic Construct
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTS

SEQ ID NO: 11 - protein, 131 aa, ARTIFICIAL SEQUENCE
  Synthetic Construct
KVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTS

SEQ ID NO: 12 - protein, 127 aa, ARTIFICIAL SEQUENCE
  Synthetic Construct
KVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADC

SEQ ID NO: 13 - protein, 20 aa, ARTIFICIAL SEQUENCE
  Synthetic Construct
GGGGSGGGGSGGGGSGGGGS

SEQ ID NO: 14 - protein, 103 aa, ARTIFICIAL SEQUENCE
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 15 - protein, 227 aa, ARTIFICIAL SEQUENCE
  Synthetic Construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 330 aa, ARTIFICIAL SEQUENCE [sequence omitted]

SEQ ID NO: 17 - protein, 123 aa, ARTIFICIAL SEQUENCE
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSGGGGSGGGGSGGGGSGGGGS

SEQ ID NO: 18 - protein, 247 aa, ARTIFICIAL SEQUENCE
  Synthetic Construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGS

SEQ ID NO: 19 - protein, 350 aa, ARTIFICIAL SEQUENCE
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGS

SEQ ID NO: 20 - protein, 342 aa, ARTIFICIAL SEQUENCE
  Synthetic Construct
IQNPDPAVYQLRDSKSSDKSVCLFTDFDSQTNVSQSKDSDVYITDKTVLDMRSMDFKSNSAVAWSNKSDFACANAFNNSIIPEDTFFPSPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGS

SEQ ID NO: 21 - protein, 107 aa, ARTIFICIAL SEQUENCE [sequence omitted]

SEQ ID NO: 22 - protein, 105 aa, ARTIFICIAL SEQUENCE
  Synthetic Construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEC

SEQ ID NO: 23 - protein, 247 aa, ARTIFICIAL SEQUENCE
  Synthetic Construct
DIVMTQTPLSLSVTPGQPASISCRSSRSLVHSRGNTYLHWYLQKPGQSPQLLIYKVSNRFIGVPDRFSGSGSGTDFTLKISRVEAEDVGVYYCSQSTHLPFTFGQGTKLEIKEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTS

SEQ ID NO: 24 - protein, 243 aa, ARTIFICIAL SEQUENCE
  Synthetic Construct
DIVMTQTPLSLSVTPGQPASISCRSSRSLVHSRGNTYLHWYLQKPGQSPQLLIYKVSNRFIGVPDRFSGSGSGTDFTLKISRVEAEDVGVYYCSQSTHLPFTFGQGTKLEIKKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTS

SEQ ID NO: 25 - protein, 211 aa, ARTIFICIAL SEQUENCE
  Synthetic Construct
DIVMTQTPLSLSVTPGQPASISCRSSRSLVHSRGNTYLHWYLQKPGQSPQLLIYKVSNRFIGVPDRFSGSGSGTDFTLKISRVEAEDVGVYYCSQSTHLPFTFGQGTKLEIKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVK

SEQ ID NO: 26 - protein, 209 aa, ARTIFICIAL SEQUENCE
  Synthetic Construct
DIVMTQTPLSLSVTPGQPASISCRSSRSLVHSRGNTYLHWYLQKPGQSPQLLIYKVSNRFIGVPDRFSGSGSGTDFTLKISRVEAEDVGVYYCSQSTHLPFTFGQGTKLEIKIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVK

SEQ ID NO: 27 - protein, 482 aa, ARTIFICIAL SEQUENCE
  Synthetic Construct
QVQLVQSGAEVKKPGSSVKVSCKASGYSFTDYHIHWVRQAPGQGLEWMGVINPMYGTTDYNQRFKGRVTITADESTSTAYMELSSLRSEDTAVYYCARYDYFTGTGVYWGQGTLVTVSSEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 28 - protein, 478 aa, ARTIFICIAL SEQUENCE
  Synthetic Construct
QVQLVQSGAEVKKPGSSVKVSCKASGYSFTDYHIHWVRQAPGQGLEWMGVINPMYGTTDYNQRFKGRVTITADESTSTAYMELSSLRSEDTAVYYCARYDYFTGTGVYWGQGTLVTVSSKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 29 - protein, 446 aa, ARTIFICIAL SEQUENCE
  Synthetic Construct
QVQLVQSGAEVKKPGSSVKVSCKASGYSFTDYHIHWVRQAPGQGLEWMGVINPMYGTTDYNQRFKGRVTITADESTSTAYMELSSLRSEDTAVYYCARYDYFTGTGVYWGQGTLVTVSSPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 30 - protein, 444 aa, ARTIFICIAL SEQUENCE
  Synthetic Construct
QVQLVQSGAEVKKPGSSVKVSCKASGYSFTDYHIHWVRQAPGQGLEWMGVINPMYGTTDYNQRFKGRVTITADESTSTAYMELSSLRSEDTAVYYCARYDYFTGTGVYWGQGTLVTVSSIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 31 - protein, 107 aa, ARTIFICIAL SEQUENCE [sequence omitted]

SEQ ID NO: 32 - protein, 120 aa, ARTIFICIAL SEQUENCE [sequence omitted]

SEQ ID NO: 33 - protein, 214 aa, ARTIFICIAL SEQUENCE
  Synthetic Construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 450 aa, ARTIFICIAL SEQUENCE
  Synthetic Construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 238 aa, ARTIFICIAL SEQUENCE
  Synthetic Construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTS

SEQ ID NO: 36 - protein, 445 aa, ARTIFICIAL SEQUENCE
  Synthetic Construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 37 - protein, 441 aa, ARTIFICIAL SEQUENCE
  Synthetic Construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSIQNPDPAVYQLRDSKSSDKSVCLFTDFDSQTNVSQSKDSDVYITDKTVLDMRSMDFKSNSAVAWSNKSDFACANAFNNSIIPEDTFFPSPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 38 - protein, 234 aa, ARTIFICIAL SEQUENCE
  Synthetic Construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADC

SEQ ID NO: 39 - protein, 107 aa, ARTIFICIAL SEQUENCE [sequence omitted]

SEQ ID NO: 40 - protein, 119 aa, ARTIFICIAL SEQUENCE [sequence omitted]

SEQ ID NO: 41 - protein, 214 aa, ARTIFICIAL SEQUENCE
  Synthetic Construct
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 449 aa, ARTIFICIAL SEQUENCE
  Synthetic Construct
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 688 aa, ARTIFICIAL SEQUENCE
  Synthetic Construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSIQNPDPAVYQLRDSKSSDKSVCLFTDFDSQTNVSQSKDSDVYITDKTVLDMRSMDFKSNSAVAWSNKSDFACANAFNNSIIPEDTFFPSPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGG

SEQ ID NO: 44 - protein, 688 aa, ARTIFICIAL SEQUENCE
  Synthetic Construct
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVGGG

SEQ ID NO: 45 - protein, 234 aa, ARTIFICIAL SEQUENCE
  Synthetic Construct
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADC

SEQ ID NO: 46 - protein, 440 aa, ARTIFICIAL SEQUENCE
  Synthetic Construct
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSIQNPDPAVYQLRDSKSSDKSVCLFTDFDSQTNVSQSKDSDVYITDKTVLDMRSMDFKSNSAVAWSNKSDFACANAFNNSIIPEDTFFPSPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 47 - protein, 688 aa, ARTIFICIAL SEQUENCE; identical to SEQ ID NO: 43
  Synthetic Construct

SEQ ID NO: 48 - protein, 688 aa, ARTIFICIAL SEQUENCE; identical to SEQ ID NO: 44
  Synthetic Construct

SEQ ID NO: 49 - protein, 683 aa, ARTIFICIAL SEQUENCE
  Synthetic Construct
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSIQNPDPAVYQLRDSKSSDKSVCLFTDFDSQTNVSQSKDSDVYITDKTVLDMRSMDFKSNSAVAWSNKSDFACANAFNNSIIPEDTFFPSPGGGGSGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 50 - protein, 683 aa, ARTIFICIAL SEQUENCE
  Synthetic Construct
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSIQNPDPAVYQLRDSKSSDKSVCLFTDFDSQTNVSQSKDSDVYITDKTVLDMRSMDFKSNSAVAWSNKSDFACANAFNNSIIPEDTFFPSPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 51 - protein, 688 aa, ARTIFICIAL SEQUENCE
  Synthetic Construct
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSIQNPDPAVYQLRDSKSSDKSVCLFTDFDSQTNVSQSKDSDVYITDKTVLDMRSMDFKSNSAVAWSNKSDFACANAFNNSIIPEDTFFPSPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGG

SEQ ID NO: 52 - protein, 688 aa, ARTIFICIAL SEQUENCE
  Synthetic Construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVGGG

SEQ ID NO: 53 - protein, 683 aa, ARTIFICIAL SEQUENCE; identical to SEQ ID NO: 49
  Synthetic Construct

SEQ ID NO: 54 - protein, 683 aa, ARTIFICIAL SEQUENCE
  Synthetic Construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSGGGGSGGGGSGGGGSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSIQNPDPAVYQLRDSKSSDKSVCLFTDFDSQTNVSQSKDSDVYITDKTVLDMRSMDFKSNSAVAWSNKSDFACANAFNNSIIPEDTFFPSPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 55 - protein, 234 aa, ARTIFICIAL SEQUENCE; identical to SEQ ID NO: 38
  Synthetic Construct

SEQ ID NO: 56 - protein, 234 aa, ARTIFICIAL SEQUENCE; identical to SEQ ID NO: 45
  Synthetic Construct

SEQ ID NO: 57 - protein, 107 aa, ARTIFICIAL SEQUENCE
  Synthetic Construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 58 - protein, 119 aa, ARTIFICIAL SEQUENCE
  Synthetic Construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 59 - protein, 214 aa, ARTIFICIAL SEQUENCE
  Synthetic Construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 448 aa, ARTIFICIAL SEQUENCE
  Synthetic Construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 61 - protein, 687 aa, ARTIFICIAL SEQUENCE
  Synthetic Construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAIQNPDPAVYQLRDSKSSDKSVCLFTDFDSQTNVSQSKDSDVYITDKTVLDMRSMDFKSNSAVAWSNKSDFACANAFNNSIIPEDTFFPSPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGG

SEQ ID NO: 62 - protein, 234 aa, ARTIFICIAL SEQUENCE
  Synthetic Construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADC

SEQ ID NO: 63 - protein, 689 aa, ARTIFICIAL SEQUENCE
  Synthetic Construct
QVQLVQSGAEVKKPGASVKVSCKASGYTFTSHWMHWVRQAPGQGLEWIGEFNPSNGRTNYNEKFKSKATMTVDTSTNTAYMELSSLRSEDTAVYYCASRDYDYDGRYFDYWGQGTLVTVSSIQNPDPAVYQLRDSKSSDKSVCLFTDFDSQTNVSQSKDSDVYITDKTVLDMRSMDFKSNSAVAWSNKSDFACANAFNNSIIPEDTFFPSPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGG

SEQ ID NO: 64 - protein, 233 aa, ARTIFICIAL SEQUENCE
  Synthetic Construct
DIQMTQSPSSLSASVGDRVTITCSASSSVTYMYWYQQKPGKAPKLLIYDTSNLASGVPSRFSGSGSGTDYTFTISSLQPEDIATYYCQQWSSHIFTFGQGTKVEIKKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADC

SEQ ID NO: 65 - protein, 687 aa, ARTIFICIAL SEQUENCE
  Synthetic Construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDGGGGSGGGGSGGGGSGGGGSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 66 - protein, 106 aa, ARTIFICIAL SEQUENCE [sequence omitted]

SEQ ID NO: 67 - protein, 121 aa, ARTIFICIAL SEQUENCE [sequence omitted]

SEQ ID NO: 68 - protein, 213 aa, ARTIFICIAL SEQUENCE
  Synthetic Construct
DIQMTQSPSSLSASVGDRVTITCSASSSVTYMYWYQQKPGKAPKLLIYDTSNLASGVPSRFSGSGSGTDYTFTISSLQPEDIATYYCQQWSSHIFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 450 aa, ARTIFICIAL SEQUENCE
  Synthetic Construct
QVQLVQSGAEVKKPGASVKVSCKASGYTFTSHWMHWVRQAPGQGLEWIGEFNPSNGRTNYNEKFKSKATMTVDTSTNTAYMELSSLRSEDTAVYYCASRDYDYDGRYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 70 - protein, 689 aa, ARTIFICIAL SEQUENCE
  Synthetic Construct
QVQLVQSGAEVKKPGASVKVSCKASGYTFTSHWMHWVRQAPGQGLEWIGEFNPSNGRTNYNEKFKSKATMTVDTSTNTAYMELSSLRSEDTAVYYCASRDYDYDGRYFDYWGQGTLVTVSSIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDGGGGSGGGGSGGGGSGGGGSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 71 - protein, 688 aa, ARTIFICIAL SEQUENCE
  Synthetic Construct
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDGGGGSGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 72 - protein, 689 aa, ARTIFICIAL SEQUENCE
  Synthetic Construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSIQNPDPAVYQLRDSKSSDKSVCLFTDFDSQTNVSQSKDSDVYITDKTVLDMRSMDFKSNSAVAWSNKSDFACANAFNNSIIPEDTFFPSPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 73 - protein, 688 aa, ARTIFICIAL SEQUENCE
  Synthetic Construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDGGGGSGGGGSGGGGSGGGGSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 74 - protein, 689 aa, ARTIFICIAL SEQUENCE
  Synthetic Construct
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSIQNPDPAVYQLRDSKSSDKSVCLFTDFDSQTNVSQSKDSDVYITDKTVLDMRSMDFKSNSAVAWSNKSDFACANAFNNSIIPEDTFFPSPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 75 - protein, 688 aa, ARTIFICIAL SEQUENCE; identical to SEQ ID NO: 71
  Synthetic Construct

SEQ ID NO: 76 - protein, 689 aa, ARTIFICIAL SEQUENCE; identical to SEQ ID NO: 72
  Synthetic Construct

SEQ ID NO: 77 - protein, 442 aa, ARTIFICIAL SEQUENCE
  Synthetic Construct
QVQLVQSGAEVKKPGASVKVSCKASGYTFTSHWMHWVRQAPGQGLEWIGEFNPSNGRTNYNEKFKSKATMTVDTSTNTAYMELSSLRSEDTAVYYCASRDYDYDGRYFDYWGQGTLVTVSSIQNPDPAVYQLRDSKSSDKSVCLFTDFDSQTNVSQSKDSDVYITDKTVLDMRSMDFKSNSAVAWSNKSDFACANAFNNSIIPEDTFFPSPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 78 - protein, 445 aa, ARTIFICIAL SEQUENCE
  Synthetic Construct
QVQLVQSGAEVKKPGSSVKVSCKASGYSFTDYHIHWVRQAPGQGLEWMGVINPMYGTTDYNQRFKGRVTITADESTSTAYMELSSLRSEDTAVYYCARYDYFTGTGVYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 79 - protein, 219 aa, ARTIFICIAL SEQUENCE
  Synthetic Construct
DIVMTQTPLSLSVTPGQPASISCRSSRSLVHSRGNTYLHWYLQKPGQSPQLLIYKVSNRFIGVPDRFSGSGSGTDFTLKISRVEAEDVGVYYCSQSTHLPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 151 aa, ARTIFICIAL SEQUENCE
  Synthetic Construct
GITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVAAFIDYKDDDDKHVHHHHHH

SEQ ID NO: 81 - protein, 25 aa, Artificial Sequence
  Synthetic Construct
ESSCDGGGGSGGGGSGGGGSGGGGS

SEQ ID NO: 82 - protein, 26 aa, Artificial Sequence
  Synthetic Construct
EPKSCDGGGGSGGGGSGGGGSGGGGS

(19) United States
(12) Patent Application Publication
(10) Pub. No.: US 2015/0183877 A1
(43) Pub. Date: Jul. 2, 2015

(54) Multi-Specific IgG-(Fab)2 Constructs Containing T-Cell Receptor Constant Domains

(71) Applicant: Eli Lilly and Company, Indianapolis, IN (US)

(72) Inventors: Stephen John Demarest, San Diego, CA (US); Xiufeng Wu, San Diego, CA (US)

(21) Appl. No.: 14/414,930

(22) PCT Filed: Jul. 15, 2013
(86) PCT No.: PCT/US2013/050436

§ 371 Date: Jan. 15, 2015

Related U.S. Application Data
(60) Provisional application No. 61/672,997, filed on Jul. 18, 2012.

(51) Int. Cl. C07K 16/28; C07K 14/725; C07K 16/40

(52) U.S. Cl. CPC C07K 16/2863; C07K 16/40; C07K 14/7051; C07K 2317/515; C07K 2317/51; C07K 2317/31; C07K 2317/524; C07K 2317/526; C07K 2317/53; C07K 2319/00; C07K 2317/64

(57) ABSTRACT

The present invention provides IgG-(Fab)2 multispecific compounds containing T-Cell Receptor (TCR) constant do mains. In addition, the present invention provides methods of making such IgG-(Fab)2 multispecific compounds.

The present disclosure relates to IgG-(Fab)₂ multi-specific compounds containing T-Cell receptor constant domains and methods of making such multi-specific compounds.

Therapeutic antibodies allow for the modulation of many disease conditions which provides benefits to patients. There are instances in which modulating more than one target could provide even greater benefit to patients. Although administration of two therapeutic antibodies is possible, practical considerations such as non-compatibility of formulations when combining multiple therapeutic antibodies into a single dosage form, and increased injections when administering multiple therapeutic antibodies as separate agents, may limit the possible benefit. A multi-specific compound could help minimize these concerns, and may also provide biological activities distinct from the combined administration of individual agents.

One of the most commonly used multi-specific compound platforms is the IgG-scFv. In this platform, an scFv is linked to each end of the heavy or light chain of an antibody. The IgG-scFv platform attempts to combine the activities of two antibodies while maintaining IgG-like pharmacokinetics and immune effector function. In scFvs, the V_{H} and V_{L} domains can exhibit attenuated stability and the exposure of hydrophobic surface area due to weaker HC/LC association can lead to aggregation.

To make IgG-scFv multispecific compounds viable, the biophysical aspects of the scFv within each particular IgG-scFv must be engineered to overcome expression, folding, affinity, solubility, and stability problems. Engineering scFvs to sufficiently overcome these issues is not straightforward, requiring significant time and resources for every multi-specific compound. As such, the IgG-scFv configuration may not always be a viable option for generating a multi-specific compound.

In Fabs, antibody heavy chain (HC) and light chain (LC) association is strong, with limited interdomain dynamics. However, manufacturing an antibody with a Fab linked to each HC or LC of the antibody (IgG-(Fab)₂) compound is challenging because the two LCs of an IgG-(Fab)₂ compound will bind heterogeneously to the two HC Fd (i.e., V_{H} and C_{H}1) regions within the compound generating unacceptable heterogeneity (sixteen HC/LC/LC pairings are possible).

The compounds of the present invention achieve specific assembly of IgG-(Fab)₂ compounds by replacing the Fab or antibody constant domains (C_{H}1/C_{L}) with T-cell receptor (TCR) constant domains. In addition, the present disclosure provides that the IgG-(Fab)₂ of the present invention contain the TCR α constant domain in the HC polypeptide to allow for proper assembly of the IgG-(Fab)₂ such that the specificities of the Fab and the antibody are maintained. The compounds of the present invention allow for the specificities and binding activities of the variable regions of two therapeutic antibodies to be combined in one compound.

While chimeric proteins containing an antibody variable region linked to a TCR constant domain have been disclosed (Kuwana, et al., Biochemical and Biophysical Research Communications, Vol. 149, No. 3, 1987 and Seimiya, et al., Journal of Biochemistry 113, 687-691, 1993), these references do not demonstrate appropriate assembly of a V_{H}-Cα and a V_{L}-Cβ polypeptides in the context of an IgG-(Fab)₂ as presently disclosed. Kuwana, et al. and Seimiya, et al. did not provide any disclosure or suggestion that using a TCR α constant domain in the heavy chain of an IgG-Fab, would allow for specific assembly of the polypeptide chains of an IgG-(Fab)₂ compound such that the binding specificities of the Fab and the antibody would be maintained.

The present disclosure provides a compound comprising a first polypeptide chain, a second polypeptide chain, and a third polypeptide chain, in which
a. the first polypeptide chain has the formula VL₁-Cβ, wherein
   i. VL₁ is a first antibody light chain variable region, and
   ii. Cβ is a T-Cell Receptor β constant domain; and
b. the second polypeptide has the formula VL₂-C_{L}, wherein
   i. V_{L2} is a second antibody light chain variable region, and
   ii. CL is a light chain κ or λ constant domain; and
c. the third polypeptide chain has a formula selected from the group consisting of

(VH₁-Cα)-X₁-(C_{H2}-C_{H3})-X₂-(VH₂-C_{H1}),
(VH₂-C_{H1})-(H-C_{H2}-C_{H3})-X₂-(VH₁-Cα),
(VH₁-Cα)-X₂-(VH₂-C_{H1})-(H-C_{H2}-C_{H3}),
and
(VH₂-C_{H1})-X₂-(VH₁-Cα)-X₁-(C_{H2}-C_{H3}),

wherein
   VH₁ is a first antibody heavy chain variable region, and
   Cα is a T-Cell Receptor α constant domain and
   X₁ is a peptide linker or is absent, and
   C_{H2}-C_{H3} are heavy chain constant domains 2 and 3, and
   H is a hinge region between C_{H1} and C_{H2}, and
   X₂ is a peptide linker or is absent, and
   VH₂ is a second antibody heavy chain variable region and
   CH1 is a heavy chain constant domain 1; and
in which the first and second polypeptide chains associate with the third polypeptide chain to form a binding site (VL₁-VH₁) for a first binding partner and a binding site for a second binding partner (VL₂-VH₂), respectively. The binding partners may be distinct antigens or proteins, or may be different positions on an antigen or protein.

The present disclosure also provides a mammalian cell containing DNA encoding three polypeptides,
a. wherein a first polypeptide has a formula the first polypeptide chain has the formula VL₁-Cβ, wherein
   VL₁ is a first antibody light chain variable region, and
   Cβ is a T-Cell Receptor β constant domain;
b. the second polypeptide has the formula VL₂-C_{L}, wherein
   VL₂ is a second antibody light chain variable region, and
   CL is a light chain κ or λ constant domain; and
c. the third polypeptide has a formula selected from the group consisting of

(VH₁-Cα)-X₁-(C_{H2}-C_{H3})-X₂-(VH₂-C_{H1}),
(VH₂-C_{H1})-(H-C_{H2}-C_{H3})-X₂-(VH₁-Cα),
(VH₁-Cα)-X₂-(VH₂-C_{H1})-(H-C_{H2}-C_{H3}),
and
(VH₂-C_{H1})-X₂-(VH₁-Cα)-X₁-(C_{H2}-C_{H3}),

wherein
   VH₁ is a first antibody heavy chain variable region, and
   Cα is a T-Cell Receptor α constant domain, and
   X₁ is a peptide linker or is absent, and
   C_{H2}-C_{H3} are heavy chain constant domains 2 and 3, and
   H is a hinge region between C_{H1} and C_{H2}, and
   X₂ is a peptide linker or is absent, and
   VH₂ is a second antibody heavy chain variable region and
   C_{H1} is a heavy chain constant domain 1.

The present disclosure provides a compound comprising an antibody and two Fabs, wherein the V_{H} containing polypeptides of the Fab are linked to the heavy chains of the antibody and wherein the C_{H1} constant regions of the antibody or Fab fragments are replaced with T-cell receptor α constant domains (Cα) and the corresponding light chain constant domains of the antibody or Fabs are replaced with T-cell receptor β constant domains (Cβ) such that the Cα and Cβ domains allow the corresponding variable regions to associate to form an antigen binding fragment and the C_{H1} and light chain constant regions allow the variable regions associate to form another antigen binding fragment.

The present disclosure provides a compound comprising an antibody and two Fabs, wherein the V_{H} containing polypeptides of the Fab are linked to the heavy chains of the antibody and wherein the C_{H1} regions of the Fab fragments are replaced with T-cell receptor α constant domains (Cα) and the constant domains of the Fab light chains are replaced with T-cell receptor β constant domains (Cβ) such that the Cα and Cβ domains associate to form part of an antigen binding fragment. The present disclosure also provides a compound comprising an antibody and two Fabs, wherein the V_{H} containing polypeptides of the Fab are linked to the heavy chains of the antibody and wherein the C_{H}1 regions of the antibody are replaced with T-cell receptor α constant domains (Cα) and the constant domains of the antibody light chain are replaced with T-cell receptor β constant domains (Cβ) such that the Cα and Cβ domains associate to form part of an antigen binding fragment.

The present disclosure provides a compound comprising three polypeptide chains, in which
a) the first polypeptide chain comprises the formula VH₁-Cα wherein
   VH₁ is a first antibody heavy chain variable region, and
   Cα is a T-cell receptor α constant domain, and
b) the second polypeptide chain comprises the formula VH₂-C_{H1}-H—C_{H2}-C_{H3} wherein
   VH₂ is a second antibody heavy chain variable region, and
   C_{H1}, C_{H2}, and C_{H3} are heavy chain constant domains 1, 2, and 3, respectively, and
   H is a hinge region, and
c) the third polypeptide chain comprises the formula VL₁-Cβ-X₁-VL₂-CL, wherein
   VL₁ is a first antibody light chain variable region,
   Cβ is a T-Cell Receptor β constant domain,
   X₁ is a linker which can be present or absent,
   VL₂ is a second antibody light chain region and
   CL is either a Cₖ or C_{λ} light chain antibody constant domain,
in which the first and second polypeptide chains associate with the third polypeptide chain to form a binding site (VL₁-VH₁) for a first binding partner and a binding site for a second binding partner (VL₂-VH₂).

The present disclosure also provides a compound comprising three polypeptide chains, wherein
a) a first polypeptide chain has the formula VH₁-C_{H1}-H-C_{H2}-C_{H3},
   wherein VH₁ is a first antibody heavy chain variable region, and
   C_{H1}, C_{H2}, and C_{H3} are heavy chain constant domains 1, 2, and 3, respectively, and
   H is a hinge region, and
b) a second polypeptide chain has the formula VL₁-CL-VL₂-Cβ, wherein
   VL₁ is a first antibody light chain variable region and
   CL is either Cₖ or C_{λ}, and
   VL₂ is a second light chain variable region, and
   Cβ is a T-Cell Receptor (3 constant domain, and
c) the third polypeptide has the formula VH₂-Cα, wherein
   VH₂ is a second antibody heavy chain variable region and
   Cα is a T-Cell Receptor α constant domain,
in which the first and third polypeptide chains associate with the second polypeptide chain to form a binding site (VL₁-VH₁) for a first binding partner and a binding site for a second binding partner (VL₂-VH₂).

The present disclosure also provides a compound comprising three polypeptide chains, wherein
a) a first polypeptide chain has the formula VL₁-CL, wherein
   VL₁ is a first antibody light chain variable region and
   CL is either Cₖ or C_{λ}, and
b) a second polypeptide chain has the formula VL₂-Cβ, wherein
   VL₂ is a second antibody light chain variable region and
   Cβ is a T-Cell Receptor (3 constant domain,
c) a third polypeptide chain has the formula VH₁-C_{H1}-X₁-VH₂-Cα, wherein
   VH₁ is a first antibody heavy chain variable region, and
   C_{H1} is a heavy chain constant domain 1, and
   X₁ is a linker, and
   VH₂ is a second antibody heavy chain variable region, and
   Cα is a T-Cell Receptor α constant domain, and
in which the first and second polypeptide chains associate with the third polypeptide chain to form a binding site (VH₁-VL₁) for a first binding partner and a binding site for a second binding partner (VH₂-VL₂).

The present disclosure also provides a compound comprising three polypeptide chains, wherein
a) a first polypeptide chain has the formula VL₁-Cβ, wherein
   VL₁ is a first antibody light chain variable region, and
   Cβ is a T-Cell Receptor β constant domain,
b) a second polypeptide has the formula VL₂-CL, wherein
   VL₂ is a second antibody light chain variable region, and
   CL is either Cₖ or C_{λ}, and
c) a third polypeptide has the formula VH₁-Cα-X₁-VH₂-C_{H1}, wherein
   VH₁ is a first antibody heavy chain variable region, and
   Cα is a T-Cell Receptor α constant domain, and
   X₁ is a linker, and
   VH₂ is a second antibody heavy chain variable, and
   C_{H1} is heavy chain constant domain 1, and
in which the first and second polypeptide chains associate with the third polypeptide chain to form a binding site (VH₁-VL₁) for a first binding partner and a binding site for a second binding partner (VH₂-VL₂).

The present invention also provides a process for producing any of the proceeding compounds comprising cultivating the mammalian cells containing DNA encoding a compound of the present invention under conditions such that the compound is expressed and recovering the compound.

### BRIEF DESCRIPTION OF THE FIGURES

FIG. 1. Schematic diagram of Format #1 of a TCR-based IgG-(Fab)₂ compound, comprising three polypeptide chains: VL₁-Cβ, VL₂-CL, and (VH₁-Cα)-X1-(C_{H2}-C_{H3})-X2-(VH₂-C_{H1}), in which VL₁ is a first antibody light chain variable region, Cβ is a T-Cell Receptor β constant domain; VL₂ is a second antibody light chain variable region, CL is a light chain κ or λ constant domain; VH₁ is a first antibody heavy chain variable region, Cα is a T-Cell Receptor α constant domain, X1 is a peptide linker or is absent, C_{H2}-C_{H3} are heavy chain constant domains 2 and 3, X₂ is a peptide linker or is absent, VH₂ is a second antibody heavy chain variable region and C_{H1} is a heavy chain constant domain 1.

FIG. 2. Schematic diagram of Format #2 of a TCR-based IgG-(Fab)₂ compound, comprising three polypeptide chains: VL₁-Cβ, VL₂-CL, and (VH₂-C_{H1})-(C_{H2}-C_{H3})-X2-(VH₁-Cα). FIG. 3. Schematic diagram of Format #3 of a TCR-based IgG-(Fab)₂ compound, comprising three polypeptide chains: VL₁-Cβ, VL₂-CL, and (VH₁-Cα)-X₂-(VH₂-C_{H1})-(C_{H2}-C_{H3}).

FIG. 4. Schematic diagram of Format #4 of a TCR-based IgG-(Fab)₂ compound, comprising three polypeptide chains: VL₁-Cβ, VL₂-CL, and (VH₂-C_{H1})-X₂-(VH₁-Cα)-X1-(C_{H1}-C_{H3}).

The general structure of an “antibody” is very well-known. For an antibody of the IgG type, there are four amino acid chains (two “heavy” chains and two “light” chains) that are cross-linked via intra- and inter-chain disulfide bonds. When expressed in certain biological systems, antibodies having unmodified human Fc sequences are glycosylated in the Fc region. Antibodies may be glycosylated at other positions as well. The subunit structures and three-dimensional configurations of antibodies are well known. Each heavy chain is comprised of an N-terminal heavy chain variable region (“V_{H}”) and a heavy chain constant region (“C_{H}”). The heavy chain constant region is comprised of three domains (C_{H}1, C_{H}2, and C_{H}3) for IgG as well as a hinge region (“hinge”) between the C_{H1} and C_{H2} domains. Each light chain is comprised of a light chain variable region (“V_{L}”) and a light chain constant region (“C_{L}”). The C_{L} may be of the kappa (“κ”) or lambda (“λ”) isotypes.

The variable regions of each light/heavy chain pair to form binding sites. The heavy chain variable region (V_{H}) and the light chain variable region (V_{L}) of the compounds of the present invention can be subdivided into regions of hypervariability, termed complementarity determining regions (“CDRs”), interspersed with regions that are more conserved, termed framework regions (“FR”). Each V_{H} and V_{L} is composed of three CDRs and four FRs, arranged from amino-terminus to carboxy-terminus in the following order: FR1, CDR1, FR2, CDR2, FR3, CDR3, FR4. Herein, the 3 CDRs of the heavy chain are referred to as “CDRH1, CDRH2, and CDRH3” and the 3 CDRs of the light chain are referred to as “CDRL1, CDRL2 and CDRL3.” The CDRs contain most of the residues which form specific interactions with the antigen. The assignment of amino acids to each domain is in accordance with well-known conventions [e.g., Kabat, “Sequences of Proteins of Immunological Interest,” National Institutes of Health, Bethesda, Md. (1991)].

An antibody may be derived from a single copy or clone (monoclonal antibody (mAb)), including e.g., any eukaryotic, prokaryotic, or phage clone. Preferably, an antibody of the present invention exists in a homogeneous or substantially homogeneous population of antibody molecules. A full-length antibody comprises full length or substantially full length constant regions, including the Fc region. An “antigen-binding fragment” of such an antibody is any shortened form of a full length antibody that comprises the antigen-binding variable regions and retains antigen-binding capability. Such shortened forms include, e.g., a Fab fragment, Fab′ fragment or F(ab′) 2 fragment that includes the CDRs or the variable regions of the antibodies disclosed.

An antibody of the present invention can be produced using techniques well known in the art, e.g., recombinant technologies, phage display technologies, synthetic technologies or combinations of such technologies or other technologies readily known in the art.

An antibody of the present invention is an engineered antibody that has been engineered to comprise framework, hinge, or constant regions derived from fully human frameworks, hinge, or constant regions containing one or more amino acid substitutions, deletions, or additions therein. Further, an antibody of the present invention is substantially non-immunogenic in humans.

A variety of different human framework sequences may be used singly or in combination as a basis for an antibody of the present invention. Preferably, the framework regions of an antibody of the present invention are of human origin or substantially human (at least 95%, 97% or 99% of human origin.) The sequences of framework regions of human origin may be obtained from The Immunoglobulin Factsbook, by Marie-Paule Lefranc, Gerard Lefranc, Academic Press 2001, ISBN 012441351.

T-cell receptors are used to recognize foreign antigenic peptides displayed by various antigen presenting cells of the immune system. Binding of T-cells to antigen-presenting cells occurs via interactions between major histocompatability complex (MHC) moieties of the antigen-presenting cells, which carry an antigenic peptide, and T-cell receptors (TCRs) on the surface of T-cells. Formation of a cell-bridging MHC-peptide-TCR complex typically results in stimulation and activation of the T-cells, a primary step of an adaptive immune response. TCRs are related to immunoglobulins in that they contain hypervariable V-class Ig-fold domains capable of generating an incredible diversity that enables them to recognize diverse peptidic antigens displayed by MHCs. Like antibody Fabs, they also contain C-class Ig-fold constant domains that help stabilize the TCR complex. However, structurally, TCR constant domains have very different primary sequences than immunoglobulin Fab constant domains (˜20% identity between the TCR α- and β-domains and IgG CH1 and CL constant domains). Additional information regarding T-cell receptors and their constant domains may be found in The T Cell Receptor Factsbook, by Marie-Paule Lefranc, Gerard Lefranc, Academic Press 2001, ISBN 0124413528.

### EXAMPLE 1

### Certain Orientations of TCR Constant Regions May Substitute for CH1/CL Regions; Variable Region-Constant Region Linker Length

To determine the ability of the TCR α- and β-constant domains (Cα and Cβ, respectively) to replace the C_{H1} of the heavy chain and light chain constant (CL) domains, varying constructs in a mammalian expression vector for transient expression in HEK293 cells are generated. Both orientations with the Cα or Cβ domains replacing the C_{H1} domain or the CL domain are constructed. For simplicity, this chimeric antibody format is called IgG-TCR. An anti-human IL-17 IgG4/κ (HC of SEQ ID NO:78 and LC of SEQ ID NO:79) antibody is utilized for generating IgG-TCR constructs. The C_{H1}/CL heterodimeric unit of the antibody is replaced using both Cα/Cβ and Cβ/Cα orientations and varying V-gene/TCR constant domain linker sequences. Each heavy chain (“HC”) construct is co-transfected with each light chain (“LC”) construct to determine whether any format will allow for assembly, binding, and stability.

The linking regions between IgG V-genes and their constant regions are shorter than the linking regions between TCR V-genes and their respective constant regions. The linker regions between the antibody V-genes and the TCR constant domains, as well as truncated versions of the α- and β-constant connector regions are generated within the IgG-TCR format. The sequences of the constructs with truncations are denoted in Table 1.

All gene constructs are synthesized using PCR-based overlapping oligonucleotide synthesis. Heavy chain genes are subcloned into a vector containing the gene sequence coding for a human IgG4 constant region resulting in the fusion products of the antibody V-genes and the TCR Cα and Cβ genes with the IgG4PAA hinge-Fc domain. Light chains are also subcloned into a vector. The vector contains a common mouse antibody LC signal sequence that is translated in-frame as part of the expressed protein and cleaved prior to secretion. The ligation mixture was used to transform E. coli strain TOP 10 competent cells (Invitrogen Corporation, Carlsbad, Calif.). Sequences are confirmed by DNA sequencing. Plasmid DNAs are used to transfect 293F cells for transient production of antibody protein.

TABLE 1
Substitution of Cα and Cβ for C_{H1} and CL in an anti-human
IL-17-IgG4; Orientations and Linker Lengths.

| Construct | C_{H1} Substitute | CL Substitute | HC | LC |
|---|---|---|---|---|
| Name | (SEQ ID NO:) | (SEQ ID NO:) | SEQ ID NO: | SEQ ID NO: |
| TCR2 | Cα | Cβ | 29 | 23 |
| | (1) | (10) | | |
| TCR4 | Cα | Cβ | 30 | 24 |
| | (2) | (11) | | |
| TCR6 | Cα | Cβ | 29 | 24 |
| | (1) | (11) | | |
| TCR8 | Cα | Cβ | 30 | 23 |
| | (2) | (10) | | |
| TCR1 | Cβ | Cα | 27 | 25 |
| | (10) | (1) | | |
| TCR3 | Cβ | Cα | 28 | 26 |
| | (11) | (2) | | |
| TCR5 | Cβ | Cα | 28 | 25 |
| | (11) | (1) | | |
| TCR7 | Cβ | Cα | 27 | 26 |
| | (10) | (2) | | |
| Anti- | — | — | 78 | 79 |
| IL17/IgG4 | | | | |

The sequence given by SEQ ID NO:2 represents a truncation of N-terminal Pro-Asn (PN) from the native human Cα sequence (given by SEQ ID NO:1). The sequence given by SEQ ID NO:11 represents a truncation of N-terminal Glu-Asp-Leu-Asn (EDLN) from the native human Cβ sequence (given by SEQ ID NO:10). The sequences of the variable regions, hinge, CH2, and CH3 are identical for these constructs. The only variation is in the CH₁/CL domains.

A vector harboring the LC/TCR DNA sequence and a vector harboring the HC/TCR DNA sequence are transfected (1:2 plasmid ratio for the HC and LC plasmids) into 293F cells using FreeStyle™ transfection reagents (Life Technologies). Transfected cells are grown at 37° C. in a 5% CO₂ incubator while shaking at 125 rpm. Secreted protein is harvested by centrifugation at >10,000 rpm for 5 min.

To purify expressed IgG-TCR constructs, supernatants are passed through 2 μm filters. 1 mL of supernatant is incubated with 100 μL resuspended, phosphate buffered saline (“PBS”)-washed Protein G magnetic beads (Millipore). Beads are washed two times with PBST (phosphate buffered saline/0.02% Tween 80) according to the manufacturer's protocols. Protein is eluted from the beads by adding 130 mL 0.01 M Acetate, pH 3.0. After harvesting, the eluants are immediately neutralized by adding 20 μL 0.1 M Tris, pH 9.0.

Each expressed IgG-TCR is evaluated by SDS-PAGE. Approximately 5 μg protein is loaded in each well of Novex® 4-20% Tris Glycine gels or 3-8% Tris-Acetate gels according to manufacturer protocols (Life Technologies). For reduced samples, 10% 0.5 M DTT in H₂O is added to the same prior to loading.

As shown in Table 2, all the proteins expressed well. However, none of the constructs having Cβ in the HC and Cα in the LC (TCR1, TCR3, TCR5, and TCR7) were assembled. Qualitative assembly was determined by SDS-PAGE based on the size of the IgG-TCR protein bands under non-reducing conditions and based on the ability to detect a LC band under reducing conditions (Table 2).

Analytical size exclusion chromatography (SEC) with in-line light scattering (SEC/LS) is performed for each sample. 30-80 μL of each sample (˜0.2-0.8 mg/mL) are injected onto a Sepax Zenix SEC 200 analytical HPLC (7.8×300 mm) column equilibrated in 10 mM phosphate, 150 mM NaCl, 0.02% NaN₃, pH 6.8, using an Agilent 1100 HPLC system (Agilent Technologies). Static light scattering data for material eluted from the SEC column are collected using a miniDAWN TREOS static light scattering detector coupled to an Optilab T-rEX in-line refractive index meter (Wyatt Technologies). UV data are analyzed using HPCHEM (Agilent). Molecular weights of the complexes are determined by their static light scattering profiles using ASTRA V (Wyatt Technologies). The SEC results (Table 2) demonstrate that the constructs aggregate to a similar level as a control IgG4 antibody.

The extent of HC/LC assembly is assessed using a kinetic Biacore experiment. Briefly, kinetic surface Plasmon resonance (SPR) experiments are performed using a Biacore 3000 (GE Healthcare). IgG-TCR proteins are captured onto CM5 sensorchip surfaces with an immobilized goat anti-human IgG-Fc polyclonal antibody (Jackson ImmunoResearch, Cat. #109-005-098). The goat polyclonal antibody immobilization is achieved by injecting the protein (at 50 μg/mL, pH 5) over an NHS/EDC activated sensorchip surface followed by blocking with ethanolamine (as described by manufacturer). The IgG or IgG-TCRs are captured onto the anti-human Fc sensorchip surface by injecting 10 μL of protein at 0.1 and 0.5 mg/mL using a 2 μL/mL flow rate. Flow rates are increased to 10 μL/min followed by secondary 30 μL injections of IL-17 (SEQ ID NO:80) at 10 and 50 nM. Following a 15 minute dissociation period, the flow rate is increased to 60 μL/min and sensorchip surfaces are regenerated using two consecutive 10 μL injections of 0.1 M glycine, pH 2.0.

The experiment measures the percent activity by relating the amount of captured IgG or IgG-TCR (based on resonance units) with the secondary response generated using a saturating level of IL-17. The percent activity of each IgG-TCR is calculated based on a comparison with the WT IgG:

$$\% Activity = \frac{( \frac{{RU}_{IL - 17}}{{RU}_{IgG \_ TCR}} )}{( \frac{{RU}_{IL - 17}}{{RU}_{IgG}} )} ,$$

where RU_{IgG}_{_{—}}_{TCR} and RU_{IgG} are the resonance units of the captured IgG-TCR or IgG, respectively, and RU_{IL-17} is the saturating level of resonance units generated by the binding of IL-17 to the pre-loaded anti-IL-17 IgG or IgG-TCR. TCR3, which had the Cα- and Cβ domains replacing the CL and CH1 domains, respectively, demonstrated little activity in the assay indicating a near complete lack of LC/HC assembly (Table 2). TCR2, TCR4, TCR6, and TCR8 (with the α- and β-constant regions replacing the CH1 and CL domains, respectively) all had activity. (Table 2).

TABLE 2
Biochemical characterization of TCR1-TCR8.

| | Expression | Apparent LC | Monomer/ | Activity by |
|---|---|---|---|---|
| Construct | Level | association by | Aggregate | kinetic |
| Name | (μg/mL) | SDS-PAGE | by SEC/LS | Biacore |
| Anti-17 | 70 | + | 64/36 | 100 |
| IgG4 | | | | |
| TCR2 | 81 | + | 63/37 | 35 |
| TCR4 | 56 | + | 67/33 | 54 |
| TCR6 | 29 | + | 64/36 | 35 |
| TCR8 | 74 | + | 65/35 | 48 |
| TCR1 | 34 | − | ~25^{a}/~75^{a} | —^{b} |
| TCR3 | 60 | − | 25/75 | 17 |
| TCR5 | 44 | − | ~25^{a}/~75^{a} | —^{b} |
| TCR7 | 27 | − | ~25^{a}/~75^{a} | —^{b} |

^{a}Estimated based on characterization of microscale purified material.
^{b}Not measured.

Thus, replacing the C_{H}1 domain of an immunoglobulin with Cα and replacing the C_{L} domain of an immunoglobulin with Cβ allows for the native-like heterotetramer formation of an IgG

### EXAMPLE 2

### IgG-TCR Hinge and LC C-Terminus Modifications

Antibody I (HC SEQ ID NO. 34; LC SEQ ID NO. 33, trastuzumab), which binds to HER2, is used for the following experiments. The variable regions of Antibody I are appended to the N-termini of TCR4 above. The IgG-TCR construct containing the Antibody I Fv is denoted as tTCR-G4(+) and has HC and LC sequences of SEQ ID NO:36 and SEQ ID NO:35, respectively. Next, the IgG4 hinge (SEQ ID NO:5) of tTCR-G4 is replaced with an IgG1 hinge (SEQ ID NO:6) resulting in tTCR-G1(+) which has a HC of SEQ ID NO:37 and a LC of SEQ ID NO:35.

To determine whether changes to Cβ in the LC can improve assembly, truncations are made in the C-terminus of Cβ. A new construct, with four residues truncated from the C-terminal sequence of Cβ is created (SEQ ID NO:12). A construct tTCR-G1(−) contains the same tTCR-G1 HC (SEQ ID NO:37) with the modified tTCR-G1 LC (SEQ ID NO:38) truncated at its C-terminus. Similarly, pTCR-G1(−) containing the Antibody II (HC SEQ ID NO:42; LC SEQ ID NO:41) variable regions (HCVR SEQ ID NO:40; LCVR SEQ ID NO:39) is constructed.

All gene constructs are synthesized using protocols similar to those described in Example 1. Protein expression and purification are performed as described in Example 1. Protein characterization is performed as described in Example 2, except that the kinetic Biacore assay used to measure IL-17 activity is replaced with a solution equilibrium assay that measures HER2 equilibrium affinity and stoichiometry. Briefly, Antibody I is directly immobilized to 12000 RUs onto an NHS/EDC activated CM5 sensor chip surface by injection of a 501.1 g/mL solution of Antibody I in 10 mM Acetate, pH 5.0. A linear RU response is observed by injecting human (h)HER-2-Fc protein (Cat. #1129-ER-050, R&D systems) at 2 μL/min at multiple concentrations between 1 and 100 nM over the sensorchip surface and monitoring the response after 80 seconds of binding. To evaluate the activity/assembly of the IgG-TCR, 20 nM hHER-2-Fc are mixed with varying concentrations (ranging from 1 nM to 100 nM) of each test article. The Antibody I sensorchip surface measures the concentration of unbound or free hHER-2-Fc ([R]_{F}) in solutions containing 20 nM hHER-2-Fc and IgG-TCR. Unbound hHER-2-Fc is equal to the total amount of receptor in solution ([R]_{T}) minus the bound concentration (Mb). The equilibrium dissociation constant, K_{D}, and binding stoichiometry, n (used to determine the % assembly), between the IgG-TCRs and hHER-2-Fc are determined using the linear relationship between [R]_{F} and linear slope of RU/time (known as the velocity or Vi) within the first 80 seconds of the experiment:

$$Vi = m \cdot [ {[ R ]}_{T} - \frac{1}{2} \{ \begin{matrix}( {n [ IgG\_TCR ]}_{T} + {[ R ]}_{T} + {K}_{D} ) - \\ \sqrt{{( {n [ IgG\_TCR ]}_{T} + {[ R ]}_{T} + {K}_{D} )}^{2} - 4 {n [ IgG\_TCR ]}_{T} + {[ R ]}_{T}}\end{matrix} \} ]$$

where m=slope of the hHER-2-Fc concentration-dependent standard curve and [IgG_TCR]_{T}=total IgG_TCR concentration.

TABLE 3
−tTCR-G1(+), tTCR-G1(−), and pTCR-G1(−) construct compositions.

| | | | HC | LC |
|---|---|---|---|---|
| Construct | | Light Chain | SEQ ID | SEQ ID |
| Name | Heavy Chain Compositions | Compositions | NO: | NO: |
| Antibody I | VH = SEQ ID NO: 32 | VL = SEQ ID NO: 31 | 34 | 33 |
| (trastuzumab) | C_{H1} = SEQ ID NO: 14 | CL = SEQ ID NO: 21 | | |
| | H = SEQ ID NO: 6 | | | |
| | CH2—CH3 = SEQ ID NO: 15 | | | |
| Antibody II | VH = SEQ ID NO: 40 | VL = SEQ ID NO: 39 | 42 | 41 |
| (pertuzumab) | C_{H1} = SEQ ID NO: 14 | CL = SEQ ID NO: 21 | | |
| | H = SEQ ID NO: 6 | | | |
| | C_{H2}—C_{H3} = SEQ ID NO: 15 | | | |
| tTCR-G1(+) | VH = SEQ ID NO: 32 | VL = SEQ ID NO: 31 | 37 | 35 |
| | Cα= SEQ ID NO: 2 | Cβ = SEQ ID NO: 11 | | |
| | X₁ = SEQ ID NO: 6 | | | |
| | C_{H2}—C_{H3} = SEQ ID NO: 15^{a} | | | |
| tTCR-G1(−) | VH = SEQ ID NO: 32 | VL = SEQ ID NO: 31 | 37 | 38 |
| | Cα = SEQ ID NO: 2 | Cβ = SEQ ID NO: 12 | | |
| | X₁ = SEQ ID NO: 6 | | | |
| | C_{H2}—C_{H3} = SEQ ID NO: 15^{a} | | | |
| pTCR-G1(−) | VH = SEQ ID NO: 40 | VL = SEQ ID NO: 39 | 46 | 45 |
| | Cα = SEQ ID NO: 2 | Cβ = SEQ ID NO: 12 | | |
| | X₁ = SEQ ID NO: 6 | | | |
| | C_{H2}—C_{H3} = SEQ ID NO: 15^{a} | | | |

^{a}The sequence for C_{H2}-C_{H3} was that of SEQ ID NO: 15, except that the terminal Lys (K) was deleted.

TABLE 4
Assembly of IgG-TCR constructs

| | | Apparent Affinity | |
|---|---|---|---|
| | Mol. | Trastuzumab or | |
| | Weight | Pertuzumab Arm | Fraction Assembly |
| Construct | (kDa) | (nM) | Relative to |
| Antibody I | 144 | n.d | 1 |
| (trastuzumab) | | | |
| Antibody II | 142 | 0.2 ± 0.2 | 1 |
| (pertuzumab) | | | |
| tTCR-G1(+) | 166 | n.d | 0.75 ± 0.2 |
| tTCR-G1(−) | 177 | 0.3 ± 0.15 | 0.96 ± 0.05 |
| pTCR-G1(−) | 180 | 0.13 ± 0.1 | 1.03 ± 0.2 |

“n.d” denotes not determined.

Truncation of the C-terminal four amino acids of Cβ results in a 5-fold average increase in protein expression as determined according to the procedure in Example 2 (data not shown). The proteins with the LC-Cβ truncation are more uniformly the expected molecular weight based on in-line light scattering measurements and do not appear to be incompletely assembled. Additionally, truncating four LC-Cβ-terminal amino acids eliminates apparent proteolysis, mis-assembly, or protein degradation that had been observed as multiple absorbance peaks eluting for non-truncated molecules (data not shown).

A Biacore-based equilibrium binding experiment between tTCR-G1(+), tTCR-G1(−), and pTCR-G1(−) proteins with the hHER-2-Fc protein demonstrates that both the tTCR-G1(−) and pTCR-G1(−) proteins are fully assembled, while the tTCR-G1(+) protein is only ˜75% assembled (Table 4). As a control, Antibody I and Antibody II are shown to block hHER-2-Fc binding to surfaces immobilized with the antibodies, but to not block each other with a stoichiometry matching the hHER-2-Fc concentration used in the assay (20 nM) (Table 4). The tTCR-G1 compounds could not inhibit hHER-2-Fc from binding an Antibody II-labeled surface and the pTCR-G1(−) compound could not inhibit hHER-2-Fc binding to an Antibody I-labeled surface, indicating that the specificity for their particular epitopes is intact. Different batches of tTCR-G1(+) appeared to give varying levels of assembly, as measured by the stoichiometry of hHER2-Fc blocking in the assay, perhaps because of differences in plasmid levels that were transfected (data not shown). The tTCR-G1(−) and pTCR-G1(−) proteins consistently block binding of 20 nM hHER2-Fc to surfaces with Antibody I and Antibody II, respectively, at 20 nM concentrations indicating they are 100% assembled. Overall, the presence of an IgG1 hinge (including the interchain cysteine that forms a disulfide with LC) and the LC-Cβ truncation results in fully assembled IgG-TCRs.

### EXAMPLE 3

### Specificity of LC and HC Assembly

To determine the extent to which introduction of TCR-constant domains into a Fab enables specific HC/LC assembly that discriminates from wild-type HC/LC assembly (i.e., HCs and LCs containing CH1 and CL domains, respectively), tTCR-G1(−) and pTCR-G1(−) HC plasmids are transfected with both their cognate Cβ constant domain containing LCs and the natural (non-TCR-domain containing) LC plasmids (Table 5). Additionally, the wild-type IgG1 Antibody I (trastuzumab) and Antibody II (pertuzumab) HC plasmids are transfected with their cognate natural LCs and their LCs containing Cβ constant domains (Table 5).

An OctetRed (ForteBio) biosensor assay is used to evaluate the specificity of the optimized Antibody I and Antibody II IgG-TCR constructs within this Example. The method includes the use of an anti-human IgG-Fc specific biosensor (ForteBio) to capture the IgG-TCR HC or wild-type IgG HC transfected in the presence of both the wild-type and TCRβ-containing LCs (Table 5) at 10 μg/mL in Octet buffer for 5 minutes. The captured IgGs or IgG-TCRs are then used to capture an anti-kappa CL domain-specific murine mAb (Sigma-Aldrich Cat. #SAB4700607) or an anti-Cβ constant domain-specific murine mAb (ThermoScientific Cat. #TCR1151) at 10 μg/mL in Octet buffer for 5 minutes. The % non-specific binding is assessed as the saturated signal of the anti-Cβ or anti-CK antibodies compared to the controls.

TABLE 5
Co-transfection of Antibody I and Antibody II IgG-TCR and IgG
HCs with both natural LCs or LCs containing TCR-β
constant domains.

| | SEQ ID NO: | | | % Non-Specific |
|---|---|---|---|---|
| Construct | HC | LC1 | LC2 | Assembly |
| tCα HC/tCβ LC (control) | 37 | 38 | — | 0 |
| tCα HC/tCβ LC/tCκ LC | 37 | 38 | 33 | 24 ± 04 |
| tG1 HC/tCκ LC (control) | 34 | 33 | — | 0 |
| tG1 HC/tCκ LC/tCβ LC | 34 | 33 | 38 | 10 ± 2 |
| pCα HC/pCβ LC (control) | 46 | 45 | — | 0 |
| pCα HC/pCβ LC/pCκ LC | 46 | 45 | 41 | 11 ± 2 |
| pG1 HC/pCκ LC (control) | 42 | 41 | — | 0 |
| pG1 HC/pCκ LC/pCβ LC | 42 | 41 | 45 | 0 ± 2 |

Wild-type IgG HCs show strong selective binding to their cognate wild-type LC when expressed in the presence of a Cβ-containing LC with the same variable domains. Additionally, Cα-containing IgG HCs show selective binding to their cognate Cβ-containing LCs when expressed in the presence of a wild-type LC.

### EXAMPLE 4

### Construction of IgG-(Fab)₂ Compounds

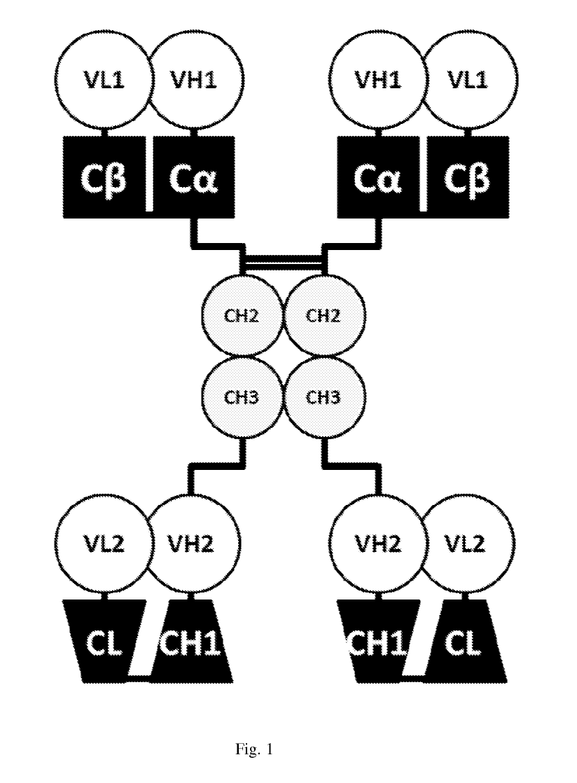

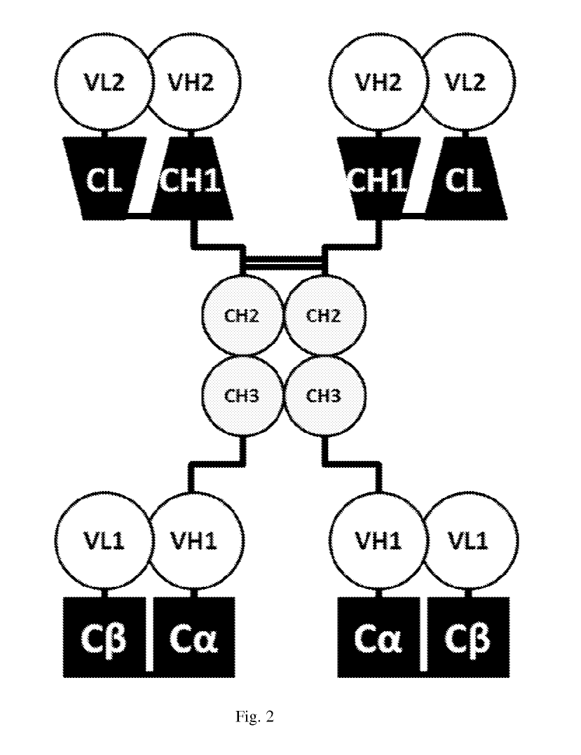

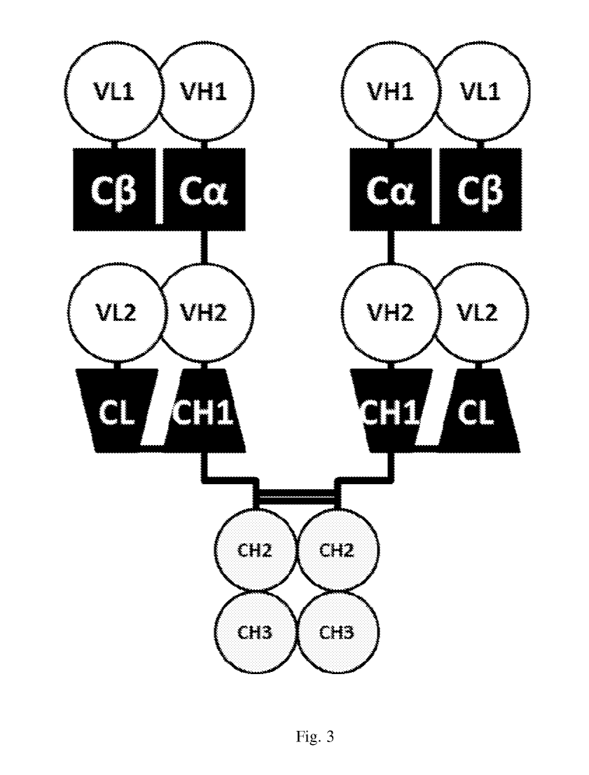

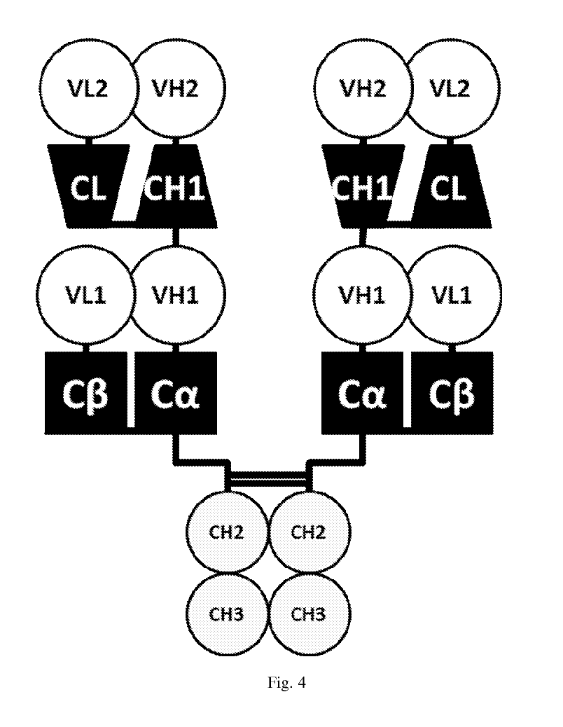

To determine whether Cα and Cβ domains can be used to generate compounds in which a Fab with one specificity is linked to an antibody with another specificity (IgG-(Fab)₂) and whether the HCs and LCs of such an IgG-(Fab)₂ can assemble appropriately to maintain their binding activities, IgG-(Fab)₂ with different Cα and Cβ configurations are constructed. In one configuration, the HC portion of the Fab is linked to the N-terminus of the HC of the antibody and in another configuration, the HC portion of the Fab is linked to the C-terminus of the antibody. For each configuration, the C_{H}1 region of the antibody or Fab is replaced with a Cα domain, while the corresponding LC constant domain is replaced with Cβ. Each of the compounds contains a HC containing both a V_{H}/C_{H}1 domain and a V_{H}/Cα domain and two LCs, one containing a V_{L}/C_{L} pair and the other containing a V_{L}/Cβ-constant domain pair. The combinations of HCs and LCs that compose each compound are listed in Table 7 and 8 and illustrated in FIGS. 1-4. All constructs fall into one of four formats, as shown below:

TABLE 6
IgG-(Fab)₂ compositions

| | FIG. | Format # |
|---|---|---|
| TCR-Fab - (CH2—CH3) - IgG-Fab | 1 | 1 |
| IgG-Fab - (CH2—CH3) - TCR-Fab | 2 | 2 |
| TCR-Fab - IgG-Fab - (CH2—CH3) | 3 | 3 |
| IgG-Fab - TCR-Fab - (CH2—CH3) | 4 | 4 |

All the methods for generation of the IgG-Fab constructs including, gene synthesis, subcloning, and plasmid preparations are described in Example 1.

TABLE 7
TCR-based IgG-(Fab)₂ Constructs

| Construct | Polypeptide Components | Format |
|---|---|---|
| Name | SEQ ID NOs: | # |
| C-tTCRpG1 | tVH-Cα-X1-(C_{H2}-C_{H3})-X2-pVH-C_{H1}^{a} | 1 |
| | 32 -2 -6 - 15 -13- 40-14 | |
| | tVL-Cβ pVL-κ | |
| | 31- 12 39-21 | |
| C-pG1tTCR | pVH-C_{H1}-(H-C_{H2}-C_{H3})-X2-tVH-Cα^{b} | 2 |
| | 40- 14-6- 15- 13- 32-2 | |
| | pVL-κ tVL-Cβ | |
| | 39-21 31-12 | |
| N-tTCRpG1 | tVH-Cα-X2-pVH-C_{H1}-(H-C_{H2}-C_{H3})^{c} | 3 |
| | 32-2 -13- 40-14 - 6- 15 | |
| | tVL-Cβ pVL-κ | |
| | 31-12 39-21 | |
| N-tTCRpG1ds | tVH-Cα-X2-pVH-C_{H1}-(H-C_{H2}-C_{H3})^{c} | 3 |
| | 32-2- 81- 40-14- 6- 15 | |
| | tVL-Cβ pVL-κ | |
| | 31-12 39-21 | |
| N-pG1tTCR | pVH-CH1-X2-tVH-Cα-X1-(C_{H2}-C_{H3})^{c} | 4 |
| | 40-14 -13- 32- 2-6- 15 | |
| | pVL-κ tVL-Cβ | |
| | 39-21 31-12 | |
| N-pG1tTCRds | pVH-C_{H1}-X2-tVH-Cα-X1-(C_{H2}-C_{H3})^{c} | 4 |
| | 40-14 -82-32 -2- 6 - 15 | |
| | pVL-κ tVL-Cβ | |
| | 39-21 31-12 | |
| C-pTCRtG1 | pVH-Cα-X1-(C_{H2}-C_{H3})-X2-tVH-C_{H1}-^{a} | 1 |
| | 40 -2 -6- 15 -13-32- 14 | |
| | pVL-Cβ tVL-κ | |
| | 39-12 31-21 | |
| C-tG1pTCR | tVH-C_{H1}-(H-C_{H2}-C_{H3})-X2-pVH-Cα^{b} | 2 |
| | 32- 14 -6- 15 -13- 40-2 | |
| | tVL-κ pVL-Cβ | |
| | 31-21 39-12 | |
| N-pTCRtG1 | pVH-Cα-X2-tVH-C_{H1}-(H-C_{H2}-C_{H3})^{c} | 3 |
| | 40-2 -13- 32-14 -6- 15 | |
| | pVL-Cβ tVL-κ | |
| | 39-12 31-21 | |
| N-pTCRtG1ds | pVH-Cα-X2-tVH-C_{H1}-(H-C_{H2}-C_{H3})^{c} | 3 |
| | 40-2- 81- 32-14 -6- 15 | |
| | pVL-Cβ tVL-κ | |
| | 39-12 31-21 | |
| N-tG1pTCR | tVH-C_{H1}-X2-pVH-Cα-X1-(C_{H2}-C_{H3})^{c} | 4 |
| | 32-14 -13-40-2- 6- 15 | |
| | tVL-κ pVL-Cβ | |
| | 31-21 39-12 | |
| N-tG1pTCRds | tVH-C_{H1}-X2-pVH-Cα-X1-(C_{H2}-C_{H3})^{c} | 4 |
| | 32-14 -82- 40-2 -6 - 15 | |
| | tVL-κ pVL-Cβ | |
| | 31-21 39-12 | |

^{a}C-terminal Glu-Pro-Lys-Ser-Cys-Asp-Gly-Gly-Gly (EPKSCDGGG) on HC.
^{b}C-terminal Glu-Ser-Ser-Cys-Asp-Val-Gly-Gly-Gly (ESSCDVGGG) on HC.
^{c}C-terminal is des-Ly (des K).

TABLE 8
Sequence compositionof IgG-(Fab)₂ compounds

| Construct | SEQ ID NOs: | | | Format |
|---|---|---|---|---|
| Name | HC | κ LC | TCR LC | # |
| C-tTCRpG1 | 47 | 41 | 55 | 1 |
| C-pG1tTCR | 48 | 41 | 55 | 2 |
| N-tTCRpG1 | 49 | 41 | 55 | 3 |
| N-tTCRpG1ds | 73 | 41 | 55 | 3 |
| N-pG1tTCR | 50 | 41 | 55 | 4 |
| N-pG1tTCRds | 74 | 41 | 55 | 4 |
| C-pTCRtG1 | 51 | 33 | 56 | 1 |
| C-tG1pTCR | 52 | 33 | 56 | 2 |
| N-pTCRtG1 | 53 | 33 | 56 | 3 |
| N-pTCRtG1ds | 71 | 33 | 56 | 3 |
| N-tG1pTCR | 54 | 33 | 56 | 4 |
| N-tG1pTCRds | 72 | 33 | 56 | 4 |
| Antibody I | 34 | 33 | — | — |
| (trastuzumab) | | | | |
| Antibody II | 42 | 41 | — | — |
| (pertuzumab) | | | | |

The following nomenclature is used when referring the IgG-Fab compounds of the present invention. “C-” or “N-” denotes an additional Fab region (either wild-type Fab or Fab containing TCR-constant domains) appended to the C- or N-terminus of the HC, respectively. “t” refers to Antibody I Fv (trastuzumab Fv), “p” refers to Antibody II (pertuzumab Fv), “G1” denotes a wild-type Fab while “TCR” denotes a Fab containing TCR-constant domains. The order with which the G1 and TCR sequences are listed within each name indicates the order within the primary sequence that the Fab regions occur. For example, C-tTCRpG1 indicates that a Fab of Antibody II (pertuzumab Fab), which has antibody C_{H1}/C_{L} domains, is linked to the C-terminal end of the HC of Antibody I (trastuzumab) whose C_{H1}/C_{L} domains are replaced by TCR Cα/Cβ, respectively.

IgG-(Fab)₂ compound characterization, including protein expression, protein purification, and in vitro biochemical are performed as described in Example 1. The binding/assembly properties of the IgG-Fab compounds are determined using the SPR-based solution equilibrium methodology described in Example 2.

TABLE 9
Characterization of the IgG-Fab recognizing two epitopes of HER2

| | | | Fraction | | Fraction |
|---|---|---|---|---|---|
| | | | Trastuzumab | Pertuzumab | Pertuzumab |
| | Mol. | Trastuzumab | Arm Assembly^{b} | arm | Arm Assembly^{b} |
| | Wt | arm apparent | (IgG_TCR n/ | apparent | (IgG_TCR n/ |
| Construct | (kDa)^{a} | affinity (nM) | Trastuz. IgG1 n) | affinity (nM) | Pertuz. IgG1 n) |
| C-tTCRpG1 | 263 | 0.09 ± 0.2 | 0.73 ± 0.2 | 0.12 ± 0.2* | 1.1 ± 0.2* |
| C-pG1tTCR | 275 | 0.21 ± 0.3 | 0.85 ± 0.2 | 1.9 ± 1.5* | 0.92 ± 0.2* |
| N-tTCRpG1 | 264 | 0.19 ± 0.3 | 0.72 ± 0.2 | 0.11 ± 0.2* | 1.2 ± 0.2* |
| N-pG1tTCR | 274 | 0.06 ± 0.2 | 0.88 ± 0.2 | 0.01 ± 0.2* | 1.15 ± 0.2* |
| C-pTCRtG1 | 256 | 0.31 ± 0.5* | 0.92 ± 0.2* | 3.2 ± 2.8 | 1.2 ± 0.2 |
| C-tG1pTCR | —^{d} | 5.0 ± 6.0* | 0.73 ± 0.3* | 0.35 ± 0.5 | 1.2 ± 0.2 |
| N-pTCRtG1 | 256 | 0.21 ± 0.5* | 1.0 ± 0.3* | 2.4 ± 2.1 | 1.2 ± 0.2 |
| N-tG1pTCR | 264 | 0.22 ± 0.5* | 1.1 ± 0.2* | 0.1 ± 1.0 | 1.0 ± 0.2 |
| N-tTCRpG1ds | 266 | 0.08 ± 0.1 | 0.87 ± 0.2 | 0.03 ± 0.1* | 1.2 ± 0.2* |
| N-pG1tTCRds | 277 | 0.04 ± 0.1 | 1.29 ± 0.2 | 0.09 ± 0.1* | 1.7 ± 0.4* |
| N-pTCRtG1ds | 256 | 0.03 ± 0.1* | 1.34 ± 0.2* | 0.08 ± 0.1 | 1.3 ± 0.2 |
| N-tG1pTCRds | 259 | 0.11 ± 0.1* | 1.39 ± 0.25* | 0.15 ± 0.1 | 1.4 ± 0.3 |

^{a}Determined by SEC with in-line static light scattering. mAb data ±5 kDa; BsAb data ±10 kda.
^{b}Determined by equilibrium solution SPR experiments.
^{c}Protein displayed varying HC/LC compositions based on SEC.
^{d}Protein displayed varying HC/LC compositions based on SEC.
*Indicates WT-IgG Fab binding stoichiometry/assembly results
“n.d” denotes not determined

The IgG-(Fab)₂ compounds could all block hHER-2-Fc from binding both surfaces (Table 9). The data suggest that four binding sites are intact.

### EXAMPLE 5

### Activity of IgG-(Fab)₂ Compounds in Cell Assays

Next, we assayed IgG-(Fab)₂ compounds for their ability to inhibit HER-2-driven tumor cell growth. The NCI-N87 (gastric) and BT474 (breast) tumor cell lines both highly over express HER-2 on their cell surfaces and have been shown to be sensitive to treatment using the trastuzumab/pertuzumab combination.

For the inhibition assays, HER-2-positive NCI-N87 (ATCC Cat. #CRL-5822) or BT474 (ATCC Cat. #HTB-20) tumor cell lines are cultured according to the guidelines provided by the ATCC. For flow cytometry, cells (˜75% confluent) are lifted from their culture flasks using cell dissociation buffer (Cat. #13151014 Life Technologies), counted, and plated in 96-well round bottom tissue culture plates at 0.5×10⁶ cells per well. Mouse Ig G1-PE (BD), Mouse anti-EGFR-PE (BD), Mouse anti-Her-2/neu-PE (BD), and Mouse anti-Her-3/erbB3-PE (BD) all are used at 20 ml/0.5×10⁶ cells. All mAbs are diluted in flow cytometry buffer (Dulbecco PBS w/2% FBS & 0.05% sodium azide & 10% NGS) and incubated for 45 minutes. The cells are centrifuged at 1500 rpm for 5 minutes at 4° C. and washed three times with flow cytometry buffer. After final wash the cells are resuspended in flow cytometry buffer containing propidium iodide (PI, Molecular Probes) to stain for dead cells. Samples are run on the LSR Fortessa acquiring with Diva software (both from Becton Dickinson) and analyzed with FloJo (version 7.6.3).

For fetal bovine serum (FBS)-mediated tumor cell proliferation experiments, NCI-N87 or BT-474 cell lines are seeded on 96-well plates at 1×10³ cells per well and precultured in RPMI-1640 medium containing 10% FBS overnight. To evaluate the impact of the IgG-(Fab)₂ compounds on cell proliferation, 100, 10, 1, and 0.1 nM solutions of each test compound (or combinations of 100, 10, 1, and 0.1 nM of each test compound) in RPMI-1640 medium containing 10% FBS are added to the cells. Alternately, 300, 30, 3, and 0.1 nM solutions of each test compound or combinations of test compounds are evaluated in the same buffer solutions. After 5 days of treatment, cell viability is determined with a Cell Titer Glo reagent (Promega). The percentage of growth inhibition is calculated according to the formula [1-(signal with mAb (or IgG-(Fab)₂))/(signal with FBS only)]*100.

To measure the levels of phosphorylated EGFR, HER-2, and HER-3 on the BT-474 cell line the following protocol is used. BT-474(breast cancer) cell lines are seeded in 12-well culture plates at 2.5×10⁵ cells per well and grown in RPMI-1640 medium containing 10% FBS overnight. The next day, cells are treated with 100 nM mAbs or IgG-(Fab)₂ for 24, 48 and 72 hours at 37° C. in 10% FBS containing medium. Cell lysates are made using cell lysis buffer (MSD Cat. #R6OTX-3), and protein concentrations are measured using BCA protein assay (Pierce). Phospho-HER-2, Phospho-HER-3 and Phospho-EGFR in cell lysates are measured using a Phospho-HER-2, Phospho-HER-3 and Phospho-EGFR multiplex MSD kit (Meso Scale Discovery). Plates are loaded with 16 μg total protein in duplicate, and incubated for 2 hours at room temperature with shaking. Next, plates are washed and detection antibody is added and incubated for 2 hours at room temperature with shaking. Plates are read on a Sector Imager6000 (Meso Scale Discovery).

TABLE 10
Inhibition of BT474 breast and NCI-N87 gastric cancer
cell lines by the IgG-(Fab)₂ compounds.

| | | | | Inhibition |
|---|---|---|---|---|
| | | | | of |
| | | | | FBS- |
| | | | | driven |
| | Inhibition of | | Inhibition of | N87 cell |
| | FBS-driven | Inhibition of | FBS-driven | prolifera- |
| | BT474 cell | FBS-driven | BT474 cell | tion |
| | proliferation | N87 cell | proliferation | (300 nM |
| | (100 nM | proliferation | (300 nM | mAb, |
| | mAb, IgG- | (100 nM mAb, | mAb, | IgG- |
| Construct | (Fab)₂) | IgG-(Fab)₂) | IgG-(Fab)₂) | (Fab)₂) |
| IgG1 control | 8 ± 7 | 9 ± 8 | −2 ± 4 | −10 ± 4 |
| Trastuzumab | 48 ± 1 | 34 ± 1 | 44 ± 11 | 28 ± 11 |
| IgG1 | | | | |
| Pertuzumab | 41 ± 2 | 46 ± 7 | 52 ± 2 | 46 ± 5 |
| IgG1 | | | | |
| Trastuzumab + | 55 ± 1 | 62 ± 4 | 59 ± 3 | 60 ± 5 |
| Pertuzumab | | | | |
| tTCR_G1(−) | −5 ± 2 | −1 ± 12 | n.d. | n.d. |
| pTCR_G1(−) | −5 ± 3 | −6 ± 11 | n.d. | n.d. |
| tTCR_G1 + | −4 ± 5 | 35 ± 3 | n.d. | n.d. |
| pTCR_G1 | | | | |
| C-tTCRpG1 | −4 ± 2 | 15 ± 17 | n.d. | n.d. |
| C-pG1tTCR | −13 ± 7 | 10 ± 16 | n.d. | n.d. |
| N-tTCRpG1 | −9 ± 3 | 11 ± 4 | n.d. | n.d. |
| N-pG1tTCR | 25 ± 1 | 35 ± 4 | 16 ± 4 | 50 ± 9 |
| C-pTCRtG1 | −10 ± 11 | −19 ± 6 | −37 ± 2 | −2 ± 6 |
| C-tG1pTCR | −10 ± 5 | −1 ± 20 | n.d. | n.d. |
| N-pTCRtG1 | 59 ± 3 | 77 ± 1 | 62 ± 1 | 82 ± 2 |
| N-tG1pTCR | −8 ± 5 | −26 ± 4 | n.d. | n.d. |
| N-pG1tTCRds | n.d. | n.d. | 33 ± 3 | 64 ± 0 |
| N-pTCRtG1ds | n.d. | n.d. | 64 ± 2 | 80 ± 1 |

“n.d” denotes not determined

As shown in Table 10: Antibody I (trastuzumab) and Antibody II (pertuzumab) individually inhibited FBS-mediated tumor cell growth of both the BT474 and NCI-N87 cell lines. In addition, the combination of Antibody I and II resulted in an increase in anti-proliferative activity. The individual IgG-TCR proteins, tTCR-G1(−) and pTCR-G1(−), and the combination of these proteins demonstrated significant decreases in anti-proliferative activity presumably due to the different Fab/hinge dynamics introduced using the subtly different connecting regions within the IgG-TCR format. Table 10 also shows that the various IgG-(Fab)₂ compounds possess a spectrum of activities on HER-2-mediated tumor cell growth ranging from antagonistic to highly-antagonistic with the N-pTCRtG1 being even more inhibitory in the NCI-N87 cell line than the Antibody I and II combination.

### EXAMPLE 6

### IgG-(Fab)₂ Combining the Specificities of the Anti-HER-2 mAb Pertuzumab and the Anti-EGFR mAbs Cetuximab and Matuzumab

To determine whether the IgG-(Fab)₂ of the present invention are able to bind to two different targets, IgG-(Fab)₂ compounds targeting EFGR and HER-2 are generated. Different constructs containing a cetuximab Fab and wild type pertuzumab (with IgG1 C_{H}1 domain and kappa C_{L} domain) as well as two additional constructs containing a matuzumab Fab and wild-type pertuzumab (with IgG1 C_{H}1 domain and kappa C_{L} domain), each in the IgG-TCR format are generated. Cetuximab is a chimeric mouse/human mAb directed against EGFR (HC of SEQ ID NO:60 and LC of SEQ ID NO:59), while matuzumab is a humanized anti-EGFR mAb (HC of SEQ ID NO:69 and LC of SEQ ID NO:68). The HCs and LCs that comprise the IgG-Fab are provided in Table 11 and 12 as are the sequences of the antibody controls and an IgG-TCR control, mTCR-G1(−). “c” refers to Antibody III (cetuximab Fv), and “m” refers to Antibody IV (matuzumab Fv) containing Fab. The remainder of the nomenclature is consistent with Example 4.

TABLE 11
Composition of the anti-HER-2/anti-EGFR IgG-Fabs
and relevant controls

| Construct | Polypeptide Components | Format |
|---|---|---|
| Name | SEQ ID NOs: | # |
| C-cTCRpG1 | cVH-Cα-X1-(C_{H2}-C_{H3})-X2-pVH-C_{H1}a | 1 |
| | 58 -2 -6- 15 13-40-14 | |
| | tVL-Cβ pVL-Cκ | |
| | 57-12 39-21 | |
| C-mTCRpG1 | mVH-Cα-X1-(C_{H2}-C_{H3})-X2-pVH-C_{H1}₋b | 1 |
| | 67 14 6- 15- 13 40- 14 | |
| | mVL-Cβ pVL-Cκ | |
| | 66-12 39-21 | |
| N-cTCRpG1 | cVH-Cα-X2-pVH-C_{H1}-(H-C_{H2}-C_{H3})c | 3 |
| | 58 -2-81- 40-14- 6- 15 | |
| | CVL-Cβ pVL-Cκ | |
| | 57-12 39-21 | |
| N-mTCRpG1 | mVH-Cα-X2-pVH-C_{H1}-(H-C_{H2}-C_{H3})c | 3 |
| | 67- 2- 81-40-14- 6- 15 | |
| | mVL-Cβ pVL-Cκ | |
| | 66-12 39-21 | |

TABLE 12
HC and LC sequence composition of the anti-HER-2/anti-EGFR IgG-
(Fab)₂ and controls

| | HC | LC | IgG-TCR LC |
|---|---|---|---|
| Construct | SEQ ID NO: | SEQ ID NO: | SEQ ID NO: |
| C-cTCRpG1 | 61 | 42 | 62 |
| C-mTCRpG1 | 63 | 42 | 64 |
| N-cTCRpG1 | 65 | 42 | 62 |
| N-mTCRpG1 | 70 | 42 | 64 |
| mTCR_G1(−) | 77 | — | 64 |
| Antibody III | 60 | 59 | — |
| (cetuximab) | | | |
| Antibody IV | 69 | 68 | — |
| (matuzumab) | | | |

The construction, expression, purification, and biophysical characterization of the anti-HER-2/anti-EGFR IgG-(Fab)₂ compounds is performed as described in the Example 4. The four IgG-(Fab)₂ compounds designed to recognize both EGFR and HER-2 are expressed and purified (by protein G magnetic beads only—no secondary purification steps) at the 2 mL and 10 mL scales.

The proteins are characterized by SDS-PAGE and analytical SEC as in Example 1.

To determine whether the IgG-(Fab)₂ compounds are able to engage both targets simultaneously, an ELISA assay is run. Specifically, clear 96-well round bottom high binding microtiter plates (Greiner) are coated overnight at 2-8° C. with 50 μL/well 1 μg/mL hEGFR-Fc (Cat. #344-ER-050, R&D systems) in a 50 mM Na₂CO₃ pH 9.4 buffer. The plate is washed 4× times with PBST and blocked with 100 μL/well casein buffer (Pierce) for 1 hr at 37° C. The plate is then washed 4× times with PBST and test compounds are added at 50 μL/well and 30 μg/mL (20 nM) and serially diluted 1:2 down the plate. The test compounds are incubated on the plate for 1 hr at 37° C. The plate is washed 4× times with PBST and 50 μL/well 0.2 μg/mL hHER-2-Histag (Sino Biologics) is added for 1 hr at 37° C. The plate is then washed 4× times with PBST followed by the addition of a 50 μL/well secondary anti-Histag-HRP antibody (PENTA-His-HRP, Qiagen) diluted 1:1000 in PBST. The secondary antibody is incubated for 1 hr at 37° C. The plate is then washed 4× times with PBST and 100 μL/well 1-component 3,3′,5,5′-Tetramethylbenzidine (TMB) substrate is added (KPL laboratories). After approximately 10 minutes, 100 μL/well 1% H₃PO₄ (in H₂O) is added to quench the reaction. Absorbance (450 nm) of every well in the plate is read using a SpectraMax UV plate reader (Molecular Devices).

TABLE 13
Characterization of TCR-based IgG-Fabs that recognize EGFR and
HER-2.

| | | | EC₅₀ |
|---|---|---|---|
| | % monomer/ | Molecular | (nM, EGFR/HER-2 |
| Construct | % aggregate | Weight (kDa) | sandwich ELISA) |
| C-cTCRpG1 | 94/6 | 268 ± 10 | 1.9 ± 1.5 |
| C-mTCRpG1 | 86/14 | 257 ± 10 | <1.0 |
| N-cTCRpG1 | 97/3 | 259 ± 10 | 3.3 ± 1.0 |
| N-mTCRpG1 | 90/10 | 254 ± 20 | 2.0 ± 2.0 |
| cetuximab | 99.5/0.5 | Not determined | No binding observed |
| IgG1 | | | |
| mTCR-G1(−) | Not determined | Not determined | No binding observed |

As shown in Table 13, the IgG-(Fab)₂ compounds have the correct molecular weight based on SDS-PAGE and analytical SEC and are primarily monodisperse with ˜3-10% soluble aggregates. Also, all four IgG-(Fab)₂ compounds are capable of binding both EGFR and HER-2 based on positive signals in the sandwich ELISA and the measured potencies are listed in Table 13.

The results of the expression and biophysical characterization indicate the IgG-(Fab)₂ compounds not only express at their expected molecular weights, but display relatively ideal starting biophysical properties. The dual-specificity binding ELISA demonstrates the ability to bind both antigens for these IgG-Fab constructs.

SEQ ID NO: 1 TCR α
PNIQNPDPAVYQLRDSKSSDKSVCLFTDFDSQTNVSQSKDSDVYITDKTV
LDMRSMDFKSNSAVAWSNKSDFACANAFNNSIIPEDTFFPSP
SEQ ID NO: 2 TCR α N-term,
IQNPDPAVYQLRDSKSSDKSVCLFTDFDSQTNVSQSKDSDVYITDKTVLD
MRSMDFKSNSAVAWSNKSDFACANAFNNSIIPEDTFFPSP
SEQ ID NO: 3 TCR α N-terminal truncated
IgG4 Fc region
IQNPDPAVYQLRDSKSSDKSVCLFTDFDSQTNVSQSKDSDVYITDKTVLD
MRSMDFKSNSAVAWSNKSDFACANAFNNSIIPEDTFFPSPESSCDVK
SEQ ID NO: 4 TCR α N term, C-term truncation +
IgG1 hinge seq
IQNPDPAVYQLRDSKSSDKSVCLFTDFDSQTNVSQSKDSDVYITDKTVLD
MRSMDFKSNSAVAWSNKSDFACANAFNNSIIPEDTFFPSPEPKSC
SEQ ID NO: 5 IgG4 hinge fragment
ESSCDVK
SEQ ID NO: 6 IgG1 hinge fragment
EPKSC
SEQ ID NO: 7 N-terminal truncated -(K) TCRα
IQNPDPAVYQLRDSKSSDKSVCLFTDFDSQTNVSQSKDSDVYITDKTVLD
MRSMDFKSNSAVAWSNKSDFACANAFNNSIIPEDTFFPSPESSCDV
SEQ ID NO: 8 N-terminal truncated -(K)TCRα + GGG
IQNPDPAVYQLRDSKSSDKSVCLFTDFDSQTNVSQSKDSDVYITDKTVLD
MRSMDFKSNSAVAWSNKSDFACANAFNNSIIPEDTFFPSPESSCDVGGG
SEQ ID NO: 9 N-terminal truncated TCRα + Linker
IQNPDPAVYQLRDSKSSDKSVCLFTDFDSQTNVSQSKDSDVYITDKTVLD
MRSMDFKSNSAVAWSNKSDFACANAFNNSIIPEDTFFPSPGGGGSGGGGS
GGGGSGGGGSGGGGS
SEQ ID NO: 10 TCRβ
EDLNKVFPPEVAVFEPSEAEISHTQKATLVCLATGFFPDHVELSWWVNGK
EVHSGVSTDPQPLKEQPALNDSRYCLSSRLRVSATFWQNPRNHFRCQVQF
YGLSENDEWTQDRAKPVTQIVSAEAWGRADCGFTS
SEQ ID NO: 11 N-term truncated TCRβ
KVFPPEVAVFEPSEAEISHTQKATLVCLATGFFPDHVELSWWVNGKEVHS
GVSTDPQPLKEQPALNDSRYCLSSRLRVSATFWQNPRNHFRCQVQFYGLS
ENDEWTQDRAKPVTQIVSAEAWGRADCGFTS
SEQ ID NO: 12 N-term, C-term truncation TCR β
KVFPPEVAVFEPSEAEISHTQKATLVCLATGFFPDHVELSWWVNGKEVHS
GVSTDPQPLKEQPALNDSRYCLSSRLRVSATFWQNPRNHFRCQVQFYGLS
ENDEWTQDRAKPVTQIVSAEAWGRADC
SEQ ID NO: 13 Linker
GGGGSGGGGSGGGGSGGGGS
SEQ ID NO: 14 CH1
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKV
SEQ ID NO: 15 CH2-CH3
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPGK
SEQ ID NO: 16 CH1-CH2-CH3
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 17 (CH1-L)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVGG
GGSGGGGSGGGGSGGGGSGGGGS
SEQ ID NO: 18 (CH2-CH3-L)
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPGKGGGGSGGGGSGGGGSGGGGS
SEQ ID NO: 19 (CH1CH2CH3-L)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGKGGGGSGGGGSGGGGSGGGGS
SEQ ID NO: 20 (A-X1-CH2CH3-X2)
IQNPDPAVYQLRDSKSSDKSVCLFTDFDSQTNVSQSKDSDVYITDKTVLD
MRSMDFKSNSAVAWSNKSDFACANAFNNSIIPEDTFFPSPEPKSCDKTHT
CPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSN
KALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSC
SVMHEALHNHYTQKSLSLSPGKGGGGSGGGGSGGGGSGGGGS
SEQ ID NO: 21 Cκ region
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC
SEQ ID NO: 22 Cλ region
GQPKAAPSVTLFPPSSEELQANKATLVCLISDFYPGAVTVAWKADSSPVK
AGVETTTPSKQSNNKYAASSYLSLTPEQWKSHRSYSCQVTHEGSTVEKTV
APTEC
SEQ ID NO: 23: anti-IL-17 LC V_{L}/Cβ
DIVMTQTPLSLSVTPGQPASISCRSSRSLVHSRGNTYLHWYLQKPGQSPQ
LLIYKVSNRFIGVPDRFSGSGSGTDFTLKISRVEAEDVGVYYCSQSTHLP
FTFGQGTKLEIKEDLNKVFPPEVAVFEPSEAEISHTQKATLVCLATGFFP
DHVELSWWVNGKEVHSGVSTDPQPLKEQPALNDSRYCLSSRLRVSATFWQ
NPRNHFRCQVQFYGLSENDEWTQDRAKPVTQIVSAEAWGRADCGFTS
SEQ ID NO: 24: anti-IL-17 LC V_{L}/Cβ_truncated
DIVMTQTPLSLSVTPGQPASISCRSSRSLVHSRGNTYLHWYLQKPGQSPQ
LLIYKVSNRFIGVPDRFSGSGSGTDFTLKISRVEAEDVGVYYCSQSTHLP
FTFGQGTKLEIKKVFPPEVAVFEPSEAEISHTQKATLVCLATGFFPDHVE
LSWWVNGKEVHSGVSTDPQPLKEQPALNDSRYCLSSRLRVSATFWQNPRN
HFRCQVQFYGLSENDEWTQDRAKPVTQIVSAEAWGRADCGFTS
SEQ ID NO: 25: anti-IL-17 LC V_{L}/Cα
DIVMTQTPLSLSVTPGQPASISCRSSRSLVHSRGNTYLHWYLQKPGQSPQ
LLIYKVSNRFIGVPDRFSGSGSGTDFTLKISRVEAEDVGVYYCSQSTHLP
FTFGQGTKLEIKPNIQNPDPAVYQLRDSKSSDKSVCLFTDFDSQTNVSQS
KDSDVYITDKTVLDMRSMDFKSNSAVAWSNKSDFACANAFNNSIIPEDTF
FPSPESSCDVK
SEQ ID NO: 26: anti-IL-17 V_{L}/Cα_truncated
DIVMTQTPLSLSVTPGQPASISCRSSRSLVHSRGNTYLHWYLQKPGQSPQ
LLIYKVSNRFIGVPDRFSGSGSGTDFTLKISRVEAEDVGVYYCSQSTHLP
FTFGQGTKLEIKIQNPDPAVYQLRDSKSSDKSVCLFTDFDSQTNVSQSKD
SDVYITDKTVLDMRSMDFKSNSAVAWSNKSDFACANAFNNSIIPEDTFFP
SPESSCDVK
SEQ ID NO: 27: anti-IL-17 HC V_{H}/Cβ/IgG4Fc
QVQLVQSGAEVKKPGSSVKVSCKASGYSFTDYHIHWVRQAPGQGLEWMGV
INPMYGTTDYNQRFKGRVTITADESTSTAYMELSSLRSEDTAVYYCARYD
YFTGTGVYWGQGTLVTVSSEDLNKVFPPEVAVFEPSEAEISHTQKATLVC
LATGFFPDHVELSWWVNGKEVHSGVSTDPQPLKEQPALNDSRYCLSSRLR
VSATFWQNPRNHFRCQVQFYGLSENDEWTQDRAKPVTQIVSAEAWGRADC
GFTSESKYGPPCPPCPAPEAAGGPSVFLFPPKPKDTLMISRTPEVTCVVV
DVSQEDPEVQFNWYVDGVEVHNAKTKPREEQFNSTYRYYSYLTYLHQDWL
NGKEYKCKVSNKGLPSSIEKTISKAKGQPREPQVYTLPPSQEEMTKNQVS
LTCLVKGFYPSDIAVEWESNGQPENNYKTTPPVLDSDGSFFLYSRLTVDK
SRWQEGNVFSCSVMHEALHNHYTQKSLSLSLG
SEQ ID NO: 28 anti-IL-17 HC V_{H}/Cβ truncated/IgG4Fc
QVQLVQSGAEVKKPGSSVKVSCKASGYSFTDYHIHWVRQAPGQGLEWMGV
INPMYGTTDYNQRFKGRVTITADESTSTAYMELSSLRSEDTAVYYCARYD
YFTGTGVYWGQGTLVTVSSKVFPPEVAVFEPSEAEISHTQKATLVCLATG
FFPDHVELSWWVNGKEVHSGVSTDPQPLKEQPALNDSRYCLSSRLRVSAT
FWQNPRNHFRCQVQFYGLSENDEWTQDRAKPVTQIVSAEAWGRADCGFTS
ESKYGPPCPPCPAPEAAGGPSVFLFPPKPKDTLMISRTPEYTCYVVDVSQ
EDPEVQFNWYYDGVEYHNAKTKPREEQFNSTYRYYSYLTYLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLG
SEQ ID NO: 29 anti-IL-17 HC V_{H}/Cα/IgG4Fc
QVQLVQSGAEVKKPGSSVKVSCKASGYSFTDYHIHWVRQAPGQGLEWMGV
INPMYGTTDYNQRFKGRVTITADESTSTAYMELSSLRSEDTAVYYCARYD
YFTGTGVYWGQGTLVTVSSPNIQNPDPAVYQLRDSKSSDKSVCLFTDFDS
QTNVSQSKDSDVYITDKTVLDMRSMDFKSNSAVAWSNKSDFACANAFNNS
IIPEDTFFPSPESSCDYKESKYGPPCPPCPAPEAAGGPSVFLFPPKPKDT
LMISRTPEYTCYVVDVSQEDPEVQFNWYYDGVEYHNAKTKPREEQFNSTY
RYYSYLTYLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLG
SEQ ID NO: 30 anti-IL-17 HC V_{H}/Cα_truncated/IgG4Fc
QVQLVQSGAEVKKPGSSVKVSCKASGYSFTDYHIHWVRQAPGQGLEWMGV
INPMYGTTDYNQRFKGRVTITADESTSTAYMELSSLRSEDTAVYYCARYD
YFTGTGVYWGQGTLVTVSSIQNPDPAVYQLRDSKSSDKSVCLFTDFDSQT
NVSQSKDSDVYITDKTYLDMRSMDFKSNSAVAWSNKSDFACANAFNNSII
PEDTFFPSPESSCDVKESKYGPPCPPCPAPEAAGGPSVFLFPPKPKDTLM
ISRTPEYTCYVVDVSQEDPEVQFNWYVDGVEVHNAKTKPREEQFNSTYRV
VSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLG
SEQ ID NO: 31 trastuzumab LCVR
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIK
SEQ ID NO: 32 trastuzumab HCVR
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSS
SEQ ID NO: 33 Trastuzumab LC
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
SEQ ID NO: 34 trastuzumab HC
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLS
PGK
SEQ ID NO: 35 anti-HER-2 trastuzumab LC V_{L}/Cβ
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKKVFPPEVAVFEPSEAEISHTQKATLVCLATGFFPDHVELSWWV
NGKEVHSGVSTDPQPLKEQPALNDSRYCLSSRLRVSATFWQNPRNHFRCQ
VQFYGLSENDEWTQDRAKPVTQIVSAEAWGRADCGFTS
SEQ ID NO: 36 anti-HER-2 trastuzumab HC V_{H}/Cα/
IgG4Fc
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSIQNPDPAVYQLRDSKSSDKSVCLFTDFDSQ
TNVSQSKDSDVYITDKTVLDMRSMDFKSNSAVAWSNKSDFACANAFNNSI
IPEDTFFPSPESSCDVKESKYGPPCPPCPAPEAAGGPSVFLFPPKPKDTL
MISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLG
SEQ ID NO: 37 anti-HER-2 trastuzumab HC V_{H}/Cα/
IgG1hinge + Fc
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSIQNPDPAVYQLRDSKSSDKSVCLFTDFDSQ
TNVSQSKDSDVYITDKTVLDMRSMDFKSNSAVAWSNKSDFACANAFNNSI
IPEDTFFPSPEPKSCDKTHTCPPCPAPELLGGPSVFLFPPKPKDTLMISR
TPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFF
LYSKLTVDKSRWQQGNVFSCSVMHEALHNHYTQKSLSLSPG
SEQ ID NO: 38 anti-HER-2 trastuzumab LC V_{L}/β
with C-terminal truncation
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKKVFPPEVAVFEPSEAEISHTQKATLVCLATGFFPDHVELSWWV
NGKEVHSGVSTDPQPLKEQPALNDSRYCLSSRLRVSATFWQNPRNHFRCQ
VQFYGLSENDEWTQDRAKPVTQIVSAEAWGRADC
SEQ ID NO: 39 pertuzumab LCVR
DIQMTQSPSSLSASVGDRVTITCKASQDVSIGVAWYQQKPGKAPKLLIYS
ASYRYTGVPSRFSGSGSGTDFTLTISSLQPEDFATYYCQQYYIYPYTFGQ
GTKVEIK
SEQ ID NO: 40 pertuzumab HCVR
EVQLVESGGGLVQPGGSLRLSCAASGFTFTDYTMDWVRQAPGKGLEWVAD
VNPNSGGSIYNQRFKGRFTLSVDRSKNTLYLQMNSLRAEDTAVYYCARNL
GPSFYFDYWGQGTLVTVSS
SEQ ID NO: 41 pertuzumab LC
DIQMTQSPSSLSASVGDRVTITCKASQDVSIGVAWYQQKPGKAPKLLIYS
ASYRYTGVPSRFSGSGSGTDFTLTISSLQPEDFATYYCQQYYIYPYTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
SEQ ID NO: 42 pertuzumab HC
EVQLVESGGGLVQPGGSLRLSCAASGFTFTDYTMDWVRQAPGKGLEWVAD
VNPNSGGSIYNQRFKGRFTLSVDRSKNTLYLQMNSLRAEDTAVYYCARNL
GPSFYFDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK
SEQ ID NO: 43 IgG_TCR BsAb HC denoted C-tTCRpG1
(C-terminal Fab fusion is wild-type
pertuzumab Fab)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSIQNPDPAVYQLRDSKSSDKSVCLFTDFDSQ
TNVSQSKDSDVYITDKTVLDMRSMDFKSNSAVAWSNKSDFACANAFNNSI
IPEDTFFPSPEPKSCDKTHTCPPCPAPELLGGPSVFLFPPKPKDTLMISR
TPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFF
LYSKLTVDKSRWQQGNVFSCSVMHEALHNHYTQKSLSLSPGKGGGGSGGG
GSGGGGSGGGGSEVQLVESGGGLVQPGGSLRLSCAASGFTFTDYTMDWVR
QAPGKGLEWVADVNPNSGGSIYNQRFKGRFTLSVDRSKNTLYLQMNSLRA
EDTAVYYCARNLGPSFYFDYWGQGTLVTVSSASTKGPSVFPLAPSSKSTS
GGTAALGCLVKDYFPEPVTVSWNSGALTSGVHTFPAVLQSSGLYSLSSVV
TVPSSSLGTQTYICNVNHKPSNTKVDKKVEPKSCDGGG
SEQ ID NO: 44 IgG_TCR BsAb HC denoted C-pG1tTCR
(C-terminal Fab fusion is IgG_TCR trastuzumab Fab)
EVQLVESGGGLVQPGGSLRLSCAASGFTFTDYTMDWVRQAPGKGLEWVAD
VNPNSGGSIYNQRFKGRFTLSVDRSKNTLYLQMNSLRAEDTAVYYCARNL
GPSFYFDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPG
KGGGGSGGGGSGGGGSGGGGSEVQLVESGGGLVQPGGSLRLSCAASGFNI
KDTYIHWVRQAPGKGLEWVARIYPTNGYTRYADSVKGRFTISADTSKNTA
YLQMNSLRAEDTAVYYCSRWGGDGFYAMDYWGQGTLVTVSSIQNPDPAVY
QLRDSKSSDKSVCLFTDFDSQTNVSQSKDSDVYITDKTVLDMRSMDFKSN
SAVAWSNKSDFACANAFNNSIIPEDTFFPSPESSCDVGGG
SEQ ID NO: 45 anti-HER-2 pertuzumab LC V_{L}/Cβ with
C-terminal truncation
DIQMTQSPSSLSASVGDRVTITCKASQDVSIGVAWYQQKPGKAPKLLIYS
ASYRYTGVPSRFSGSGSGTDFTLTISSLQPEDFATYYCQQYYIYPYTFGQ
GTKVEIKKVFPPEVAVFEPSEAEISHTQKATLVCLATGFFPDHVELSWWV
NGKEVHSGVSTDPQPLKEQPALNDSRYCLSSRLRVSATFWQNPRNHFRCQ
VQFYGLSENDEWTQDRAKPVTQIVSAEAWGRADC
SEQ ID NO: 46 anti-HER-2 pertuzumab HC V_{H}/Cα/
IgG1hinge + Fc
EVQLVESGGGLVQPGGSLRLSCAASGFTFTDYTMDWVRQAPGKGLEWVAD
VNPNSGGSIYNQRFKGRFTLSVDRSKNTLYLQMNSLRAEDTAVYYCARNL
GPSFYFDYWGQGTLVTVSSIQNPDPAVYQLRDSKSSDKSVCLFTDFDSQT
NVSQSKDSDVYITDKTVLDMRSMDFKSNSAVAWSNKSDFACANAFNNSII
PEDTFFPSPEPKSCDKTHTCPPCPAPELLGGPSVFLFPPKPKDTLMISRT
PEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFL
YSKLTVDKSRWQQGNVFSCSVMHEALHNHYTQKSLSLSPG
SEQ ID NO: 47 C-tTCRpG1 HC
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSIQNPDPAVYQLRDSKSSDKSVCLFTDFDSQ
TNVSQSKDSDVYITDKTVLDMRSMDFKSNSAVAWSNKSDFACANAFNNSI
IPEDTFFPSPEPKSCDKTHTCPPCPAPELLGGPSVFLFPPKPKDTLMISR
TPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFF
LYSKLTVDKSRWQQGNVFSCSVMHEALHNHYTQKSLSLSPGKGGGGSGGG
GSGGGGSGGGGSEVQLVESGGGLVQPGGSLRLSCAASGFTFTDYTMDWVR
QAPGKGLEWVADVNPNSGGSIYNQRFKGRFTLSVDRSKNTLYLQMNSLRA
EDTAVYYCARNLGPSFYFDYWGQGTLVTVSSASTKGPSVFPLAPSSKSTS
GGTAALGCLVKDYFPEPVTVSWNSGALTSGVHTFPAVLQSSGLYSLSSVV
TVPSSSLGTQTYICNVNHKPSNTKVDKKVEPKSCDGGG
SEQ ID NO: 48 C-pG1tTCR HC
EVQLVESGGGLVQPGGSLRLSCAASGFTFTDYTMDWVRQAPGKGLEWVAD
VNPNSGGSIYNQRFKGRFTLSVDRSKNTLYLQMNSLRAEDTAVYYCARNL
GPSFYFDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGKG
GGGSGGGGSGGGGSGGGGSEVQLVESGGGLVQPGGSLRLSCAASGFNIKD
TYIHWVRQAPGKGLEWVARIYPTNGYTRYADSVKGRFTISADTSKNTAYL
QMNSLRAEDTAVYYCSRWGGDGFYAMDYWGQGTLVTVSSIQNPDPAVYQL
RDSKSSDKSVCLFTDFDSQTNVSQSKDSDVYITDKTVLDMRSMDFKSNSA
VAWSNKSDFACANAFNNSIIPEDTFFPSPESSCDVGGG
SEQ ID NO: 49 N-tTCRpG1 HC
EVQLVESGGGLVQPGGSLRLSCAASGFTFTDYTMDWVRQAPGKGLEWVAD
VNPNSGGSIYNQRFKGRFTLSVDRSKNTLYLQMNSLRAEDTAVYYCARNL
GPSFYFDYWGQGTLVTVSSIQNPDPAVYQLRDSKSSDKSVCLFTDFDSQT
NVSQSKDSDVYITDKTVLDMRSMDFKSNSAVAWSNKSDFACANAFNNSII
PEDTFFPSPGGGGSGGGGSGGGGSGGGGSGGGGSEVQLVESGGGLVQPGG
SLRLSCAASGFNIKDTYIHWVRQAPGKGLEWVARIYPTNGYTRYADSVKG
RFTISADTSKNTAYLQMNSLRAEDTAVYYCSRWGGDGFYAMDYWGQGTLV
TVSSASTKGPSVFPLAPSSKSTSGGTAALGCLVKDYFPEPVTVSWNSGAL
TSGVHTFPAVLQSSGLYSLSSVVTVPSSSLGTQTYICNVNHKPSNTKVDK
KVEPKSCDKTHTCPPCPAPELLGGPSVFLFPPKPKDTLMISRTPEVTCVV
VDVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTVD
KSRWQQGNVFSCSVMHEALHNHYTQKSLSLSPG
SEQ ID NO: 50 N-pG1tTCR HC
EVQLVESGGGLVQPGGSLRLSCAASGFTFTDYTMDWVRQAPGKGLEWVAD
VNPNSGGSIYNQRFKGRFTLSVDRSKNTLYLQMNSLRAEDTAVYYCARNL
GPSFYFDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVGGGGSGGGGSGGGGSGGGGSGGGGSEVQLVESG
GGLVQPGGSLRLSCAASGFNIKDTYIHWVRQAPGKGLEWVARIYPTNGYT
RYADSVKGRFTISADTSKNTAYLQMNSLRAEDTAVYYCSRWGGDGFYAMD
YWGQGTLVTVSSIQNPDPAVYQLRDSKSSDKSVCLFTDFDSQTNVSQSKD
SDVYITDKTVLDMRSMDFKSNSAVAWSNKSDFACANAFNNSIIPEDTFFP
SPEPKSCDKTHTCPPCPAPELLGGPSVFLFPPKPKDTLMISRTPEVTCVV
VDVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTVD
KSRWQQGNVFSCSVMHEALHNHYTQKSLSLSPG
SEQ ID NO: 51 C-pTCRtG1 HC
EVQLVESGGGLVQPGGSLRLSCAASGFTFTDYTMDWVRQAPGKGLEWVAD
VNPNSGGSIYNQRFKGRFTLSVDRSKNTLYLQMNSLRAEDTAVYYCARNL
GPSFYFDYWGQGTLVTVSSIQNPDPAVYQLRDSKSSDKSVCLFTDFDSQT
NVSQSKDSDVYITDKTVLDMRSMDFKSNSAVAWSNKSDFACANAFNNSII
PEDTFFPSPEPKSCDKTHTCPPCPAPELLGGPSVFLFPPKPKDTLMISRT
PEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFL
YSKLTVDKSRWQQGNVFSCSVMHEALHNHYTQKSLSLSPGKGGGGSGGGG
SGGGGSGGGGSEVQLVESGGGLVQPGGSLRLSCAASGFNIKDTYIHWVRQ
APGKGLEWVARIYPTNGYTRYADSVKGRFTISADTSKNTAYLQMNSLRAE
DTAVYYCSRWGGDGFYAMDYWGQGTLVTVSSASTKGPSVFPLAPSSKSTS
GGTAALGCLVKDYFPEPVTVSWNSGALTSGVHTFPAVLQSSGLYSLSSVV
TVPSSSLGTQTYICNVNHKPSNTKVDKKVEPKSCDGGG
SEQ ID NO: 52 C-tG1pTCR HC
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK
GGGGSGGGGSGGGGSGGGGSEVQLVESGGGLVQPGGSLRLSCAASGFTFT
DYTMDWVRQAPGKGLEWVADVNPNSGGSIYNQRFKGRFTLSVDRSKNTLY
LQMNSLRAEDTAVYYCARNLGPSFYFDYWGQGTLVTVSSIQNPDPAVYQL
RDSKSSDKSVCLFTDFDSQTNVSQSKDSDVYITDKTVLDMRSMDFKSNSA
VAWSNKSDFACANAFNNSIIPEDTFFPSPESSCDVGGG
SEQ ID NO: 53 N-pTCRtG1 HC
EVQLVESGGGLVQPGGSLRLSCAASGFTFTDYTMDWVRQAPGKGLEWVAD
VNPNSGGSIYNQRFKGRFTLSVDRSKNTLYLQMNSLRAEDTAVYYCARNL
GPSFYFDYWGQGTLVTVSSIQNPDPAVYQLRDSKSSDKSVCLFTDFDSQT
NVSQSKDSDVYITDKTVLDMRSMDFKSNSAVAWSNKSDFACANAFNNSII
PEDTFFPSPGGGGSGGGGSGGGGSGGGGSGGGGSEVQLVESGGGLVQPGG
SLRLSCAASGFNIKDTYIHWVRQAPGKGLEWVARIYPTNGYTRYADSVKG
RFTISADTSKNTAYLQMNSLRAEDTAVYYCSRWGGDGFYAMDYWGQGTLV
TVSSASTKGPSVFPLAPSSKSTSGGTAALGCLVKDYFPEPVTVSWNSGAL
TSGVHTFPAVLQSSGLYSLSSVVTVPSSSLGTQTYICNVNHKPSNTKVDK
KVEPKSCDKTHTCPPCPAPELLGGPSVFLFPPKPKDTLMISRTPEVTCVV
VDVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTVD
KSRWQQGNVFSCSVMHEALHNHYTQKSLSLSPG
SEQ ID NO: 54 N-tG1pTCR HC
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVGGGGSGGGGSGGGGSGGGGSGGGGSEVQLVES
GGGLVQPGGSLRLSCAASGFTFTDYTMDWVRQAPGKGLEWVADVNPNSGG
SIYNQRFKGRFTLSVDRSKNTLYLQMNSLRAEDTAVYYCARNLGPSFYFD
YWGQGTLVTVSSIQNPDPAVYQLRDSKSSDKSVCLFTDFDSQTNVSQSKD
SDVYITDKTVLDMRSMDFKSNSAVAWSNKSDFACANAFNNSIIPEDTFFP
SPEPKSCDKTHTCPPCPAPELLGGPSVFLFPPKPKDTLMISRTPEVTCVV
VDVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTVD
KSRWQQGNVFSCSVMHEALHNHYTQKSLSLSPG
SEQ ID NO: 55 Trastuzumab V_{L}/TCR-β LC
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKKVFPPEVAVFEPSEAEISHTQKATLVCLATGFFPDHVELSWWV
NGKEVHSGVSTDPQPLKEQPALNDSRYCLSSRLRVSATFWQNPRNHFRCQ
VQFYGLSENDEWTQDRAKPVTQIVSAEAWGRADC
SEQ ID NO: 56 Pertuzumab V_{L}/TCR-β LC
DIQMTQSPSSLSASVGDRVTITCKASQDVSIGVAWYQQKPGKAPKLLIYS
ASYRYTGVPSRFSGSGSGTDFTLTISSLQPEDFATYYCQQYYIYPYTFGQ
GTKVEIKKVFPPEVAVFEPSEAEISHTQKATLVCLATGFFPDHVELSWWV
NGKEVHSGVSTDPQPLKEQPALNDSRYCLSSRLRVSATFWQNPRNHFRCQ
VQFYGLSENDEWTQDRAKPVTQIVSAEAWGRADC
SEQ ID NO: 57 cetuximab LCVR
DILLTQSPVILSVSPGERVSFSCRASQSIGTNIHWYQQRTNGSPRLLIKY
ASESISGIPSRFSGSGSGTDFTLSINSVESEDIADYYCQQNNNWPTTFGA
GTKLELK
SEQ ID NO: 58 cetuximab HCVR
QVQLKQSGPGLVQPSQSLSITCTVSGFSLTNYGVHWVRQSPGKGLEWLGV
IWSGGNTDYNTPFTSRLSINKDNSKSQVFFKMNSLQSNDTAIYYCARALT
YYDYEFAYWGQGTLVTVSA
SEQ ID NO: 59 Cetuximab LC
DILLTQSPVILSVSPGERVSFSCRASQSIGTNIHWYQQRTNGSPRLLIKY
ASESISGIPSRFSGSGSGTDFTLSINSVESEDIADYYCQQNNNWPTTFGA
GTKLELKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
SEQ ID NO: 60 Cetuximab HC
QVQLKQSGPGLVQPSQSLSITCTVSGFSLTNYGVHWVRQSPGKGLEWLGV
IWSGGNTDYNTPFTSRLSINKDNSKSQVFFKMNSLQSNDTAIYYCARALT
YYDYEFAYWGQGTLVTVSAASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPG
SEQ ID NO: 61 C-cTCRpG1 HC
QVQLKQSGPGLVQPSQSLSITCTVSGFSLTNYGVHWVRQSPGKGLEWLGV
IWSGGNTDYNTPFTSRLSINKDNSKSQVFFKMNSLQSNDTAIYYCARALT
YYDYEFAYWGQGTLVTVSAIQNPDPAVYQLRDSKSSDKSVCLFTDFDSQT
NVSQSKDSDVYITDKTVLDMRSMDFKSNSAVAWSNKSDFACANAFNNSII
PEDTFFPSPEPKSCDKTHTCPPCPAPELLGGPSVFLFPPKPKDTLMISRT
PEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFL
YSKLTVDKSRWQQGNVFSCSVMHEALHNHYTQKSLSLSPGKGGGGSGGGG
SGGGGSGGGGSEVQLVESGGGLVQPGGSLRLSCAASGFTFTDYTMDWVRQ
APGKGLEWVADVNPNSGGSIYNQRFKGRFTLSVDRSKNTLYLQMNSLRAE
DTAVYYCARNLGPSFYFDYWGQGTLVTVSSASTKGPSVFPLAPSSKSTSG
GTAALGCLVKDYFPEPVTVSWNSGALTSGVHTFPAVLQSSGLYSLSSVVT
VPSSSLGTQTYICNVNHKPSNTKVDKKVEPKSCDGGG
SEQ ID NO: 62 Cetuximab V_{L}/TCR-LC
DILLTQSPVILSVSPGERVSFSCRASQSIGTNIHWYQQRTNGSPRLLIKY
ASESISGIPSRFSGSGSGTDFTLSINSVESEDIADYYCQQNNNWPTTFGA
GTKLELKKVFPPEVAVFEPSEAEISHTQKATLVCLATGFFPDHVELSWWV
NGKEVHSGVSTDPQPLKEQPALNDSRYCLSSRLRVSATFWQNPRNHFRCQ
VQFYGLSENDEWTQDRAKPVTQIVSAEAWGRADC
SEQ ID NO: 63 C-mTCRpG1 HC
QVQLVQSGAEVKKPGASVKVSCKASGYTFTSHWMHWVRQAPGQGLEWIGE
FNPSNGRTNYNEKFKSKATMTVDTSTNTAYMELSSLRSEDTAVYYCASRD
YDYDGRYFDYWGQGTLVTVSSIQNPDPAVYQLRDSKSSDKSVCLFTDFDS
QTNVSQSKDSDVYITDKTVLDMRSMDFKSNSAVAWSNKSDFACANAFNNS
IIPEDTFFPSPEPKSCDKTHTCPPCPAPELLGGPSVFLFPPKPKDTLMIS
RTPEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGSF
FLYSKLTVDKSRWQQGNVFSCSVMHEALHNHYTQKSLSLSPGKGGGGSGG
GGSGGGGSGGGGSEVQLVESGGGLVQPGGSLRLSCAASGFTFTDYTMDWV
RQAPGKGLEWVADVNPNSGGSIYNQRFKGRFTLSVDRSKNTLYLQMNSLR
AEDTAVYYCARNLGPSFYFDYWGQGTLVTVSSASTKGPSVFPLAPSSKST
SGGTAALGCLVKDYFPEPVTVSWNSGALTSGVHTFPAVLQSSGLYSLSSV
VTVPSSSLGTQTYICNVNHKPSNTKVDKKVEPKSCDGGG
SEQ ID NO: 64 matuzumab V_{L}/TCR-LC
DIQMTQSPSSLSASVGDRVTITCSASSSVTYMYWYQQKPGKAPKLLIYDT
SNLASGVPSRFSGSGSGTDYTFTISSLQPEDIATYYCQQWSSHIFTFGQG
TKVEIKKVFPPEVAVFEPSEAEISHTQKATLVCLATGFFPDHVELSWWVN
GKEVHSGVSTDPQPLKEQPALNDSRYCLSSRLRVSATFWQNPRNHFRCQV
QFYGLSENDEWTQDRAKPVTQIVSAEAWGRADC
SEQ ID NO: 65 N-cTCRpG1ds HC
QVQLKQSGPGLVQPSQSLSITCTVSGFSLTNYGVHWVRQSPGKGLEWLGV
IWSGGNTDYNTPFTSRLSINKDNSKSQVFFKMNSLQSNDTAIYYCARALT
YYDYEFAYWGQGTLVTVSAIQNPDPAVYQLRDSKSSDKSVCLFTDFDSQT
NVSQSKDSDVYITDKTVLDMRSMDFKSNSAVAWSNKSDFACANAFNNSII
PEDTFFPSPESSCDGGGGSGGGGSGGGGSGGGGSGGGGSEVQLVESGGGL
VQPGGSLRLSCAASGFTFTDYTMDWVRQAPGKGLEWVADVNPNSGGSIYN
QRFKGRFTLSVDRSKNTLYLQMNSLRAEDTAVYYCARNLGPSFYFDYWGQ
GTLVTVSSASTKGPSVFPLAPSSKSTSGGTAALGCLVKDYFPEPVTVSWN
SGALTSGVHTFPAVLQSSGLYSLSSVVTVPSSSLGTQTYICNVNHKPSNT
KVDKKVEPKSCDKTHTCPPCPAPELLGGPSVFLFPPKPKDTLMISRTPEV
TCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDELT
KNQVSLTCLVKGFYPSDIAVEWESNGQPENNYKTTPPVLDSDGSFFLYSK
LTVDKSRWQQGNVFSCSVMHEALHNHYTQKSLSLSPG
SEQ ID NO: 66 matuzumab LCVR
DIQMTQSPSSLSASVGDRVTITCSASSSVTYMYWYQQKPGKAPKLLIYDT
SNLASGVPSRFSGSGSGTDYTFTISSLQPEDIATYYCQQWSSHIFTFGQG
TKVEIK
SEQ ID NO: 67 matuzumab HCVR
QVQLVQSGAEVKKPGASVKVSCKASGYTFTSHWMHWVRQAPGQGLEWIGE
FNPSNGRTNYNEKFKSKATMTVDTSTNTAYMELSSLRSEDTAVYYCASRD
YDYDGRYFDYWGQGTLVTVSS
SEQ ID NO: 68 matuzumab LC
DIQMTQSPSSLSASVGDRVTITCSASSSVTYMYWYQQKPGKAPKLLIYDT
SNLASGVPSRFSGSGSGTDYTFTISSLQPEDIATYYCQQWSSHIFTFGQG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC
SEQ ID NO: 69 matuzumab HC
QVQLVQSGAEVKKPGASVKVSCKASGYTFTSHWMHWVRQAPGQGLEWIGE
FNPSNGRTNYNEKFKSKATMTVDTSTNTAYMELSSLRSEDTAVYYCASRD
YDYDGRYFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPG
SEQ ID NO: 70 N-mTCRpG1ds HC
QVQLVQSGAEVKKPGASVKVSCKASGYTFTSHWMHWVRQAPGQGLEWIGE
FNPSNGRTNYNEKFKSKATMTVDTSTNTAYMELSSLRSEDTAVYYCASRD
YDYDGRYFDYWGQGTLVTVSSIQNPDPAVYQLRDSKSSDKSVCLFTDFDS
QTNVSQSKDSDVYITDKTVLDMRSMDFKSNSAVAWSNKSDFACANAFNNS
IIPEDTFFPSPESSCDGGGGSGGGGSGGGGSGGGGSGGGGSEVQLVESGG
GLVQPGGSLRLSCAASGFTFTDYTMDWVRQAPGKGLEWVADVNPNSGGSI
YNQRFKGRFTLSVDRSKNTLYLQMNSLRAEDTAVYYCARNLGPSFYFDYW
GQGTLVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRTP
EVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLY
SKLTVDKSRWQQGNVFSCSVMHEALHNHYTQKSLSLSPG
SEQ ID NO: 71 N-pTCRtG1ds HC
EVQLVESGGGLVQPGGSLRLSCAASGFTFTDYTMDWVRQAPGKGLEWVAD
VNPNSGGSIYNQRFKGRFTLSVDRSKNTLYLQMNSLRAEDTAVYYCARNL
GPSFYFDYWGQGTLVTVSSIQNPDPAVYQLRDSKSSDKSVCLFTDFDSQT
NVSQSKDSDVYITDKTVLDMRSMDFKSNSAVAWSNKSDFACANAFNNSII
PEDTFFPSPESSCDGGGGSGGGGSGGGGSGGGGSGGGGSEVQLVESGGGL
VQPGGSLRLSCAASGFNIKDTYIHWVRQAPGKGLEWVARIYPTNGYTRYA
DSVKGRFTISADTSKNTAYLQMNSLRAEDTAVYYCSRWGGDGFYAMDYWG
QGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSN
TKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLYS
KLTVDKSRWQQGNVFSCSVMHEALHNHYTQKSLSLSPG
SEQ ID NO: 72 N-tG1pTCRds HC
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDGGGGSGGGGSGGGGSGGGGSGGGGSE
VQLVESGGGLVQPGGSLRLSCAASGFTFTDYTMDWVRQAPGKGLEWVADV
NPNSGGSIYNQRFKGRFTLSVDRSKNTLYLQMNSLRAEDTAVYYCARNLG
PSFYFDYWGQGTLVTVSSIQNPDPAVYQLRDSKSSDKSVCLFTDFDSQTN
VSQSKDSDVYITDKTVLDMRSMDFKSNSAVAWSNKSDFACANAFNNSIIP
EDTFFPSPEPKSCDKTHTCPPCPAPELLGGPSVFLFPPKPKDTLMISRTP
EVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLY
SKLTVDKSRWQQGNVFSCSVMHEALHNHYTQKSLSLSPG
SEQ ID NO: 73 N-tTCRpG1ds HC
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSIQNPDPAVYQLRDSKSSDKSVCLFTDFDSQ
TNVSQSKDSDVYITDKTVLDMRSMDFKSNSAVAWSNKSDFACANAFNNSI
IPEDTFFPSPESSCDGGGGSGGGGSGGGGSGGGGSGGGGSEVQLVESGGG
LVQPGGSLRLSCAASGFTFTDYTMDWVRQAPGKGLEWVADVNPNSGGSIY
NQRFKGRFTLSVDRSKNTLYLQMNSLRAEDTAVYYCARNLGPSFYFDYWG
QGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSN
TKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLYS
KLTVDKSRWQQGNVFSCSVMHEALHNHYTQKSLSLSPG
SEQ ID NO: 74 N-pG1tTCRds HC
EVQLVESGGGLVQPGGSLRLSCAASGFTFTDYTMDWVRQAPGKGLEWVAD
VNPNSGGSIYNQRFKGRFTLSVDRSKNTLYLQMNSLRAEDTAVYYCARNL
GPSFYFDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDGGGGSGGGGSGGGGSGGGGSGGGGSEV
QLVESGGGLVQPGGSLRLSCAASGFNIKDTYIHWVRQAPGKGLEWVARIY
PTNGYTRYADSVKGRFTISADTSKNTAYLQMNSLRAEDTAVYYCSRWGGD
GFYAMDYWGQGTLVTVSSIQNPDPAVYQLRDSKSSDKSVCLFTDFDSQTN
VSQSKDSDVYITDKTVLDMRSMDFKSNSAVAWSNKSDFACANAFNNSIIP
EDTFFPSPEPKSCDKTHTCPPCPAPELLGGPSVFLFPPKPKDTLMISRTP
EVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLY
SKLTVDKSRWQQGNVFSCSVMHEALHNHYTQKSLSLSPG
SEQ ID NO: 75 N-pTCRtG1ds HC
EVQLVESGGGLVQPGGSLRLSCAASGFTFTDYTMDWVRQAPGKGLEWVAD
VNPNSGGSIYNQRFKGRFTLSVDRSKNTLYLQMNSLRAEDTAVYYCARNL
GPSFYFDYWGQGTLVTVSSIQNPDPAVYQLRDSKSSDKSVCLFTDFDSQT
NVSQSKDSDVYITDKTVLDMRSMDFKSNSAVAWSNKSDFACANAFNNSII
PEDTFFPSPESSCDGGGGSGGGGSGGGGSGGGGSGGGGSEVQLVESGGGL
VQPGGSLRLSCAASGFNIKDTYIHWVRQAPGKGLEWVARIYPTNGYTRYA
DSVKGRFTISADTSKNTAYLQMNSLRAEDTAVYYCSRWGGDGFYAMDYWG
QGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSN
TKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLY
SKLTVDKSRWQQGNVFSCSVMHEALHNHYTQKSLSLSPG
SEQ ID NO: 76 N-tG1pTCRds HC
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDGGGGSGGGGSGGGGSGGGGSGGGGSE
VQLVESGGGLVQPGGSLRLSCAASGFTFTDYTMDWVRQAPGKGLEWVADV
NPNSGGSIYNQRFKGRFTLSVDRSKNTLYLQMNSLRAEDTAVYYCARNLG
PSFYFDYWGQGTLVTVSSIQNPDPAVYQLRDSKSSDKSVCLFTDFDSQTN
VSQSKDSDVYITDKTVLDMRSMDFKSNSAVAWSNKSDFACANAFNNSIIP
EDTFFPSPEPKSCDKTHTCPPCPAPELLGGPSVFLFPPKPKDTLMISRTP
EVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLY
SKLTVDKSRWQQGNVFSCSVMHEALHNHYTQKSLSLSPG
SEQ ID NO: 77 mTCR_G1(-)HC (IgG-TCR)
QVQLVQSGAEVKKPGASVKVSCKASGYTFTSHWMHWVRQAPGQGLEWIGE
FNPSNGRTNYNEKFKSKATMTVDTSTNTAYMELSSLRSEDTAVYYCASRD
YDYDGRYFDYWGQGTLVTVSSIQNPDPAVYQLRDSKSSDKSVCLFTDFDS
QTNVSQSKDSDVYITDKTVLDMRSMDFKSNSAVAWSNKSDFACANAFNNS
IIPEDTFFPSPEPKSCDKTHTCPPCPAPELLGGPSVFLFPPKPKDTLMIS
RTPEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGSF
FLYSKLTVDKSRWQQGNVFSCSVMHEALHNHYTQKSLSLSPG
SEQ ID NO: 78 IL-17 IgG4 antibody HC
QVQLVQSGAEVKKPGSSVKVSCKASGYSFTDYHIHWVRQAPGQGLEWMGV
INPMYGTTDYNQRFKGRVTITADESTSTAYMELSSLRSEDTAVYYCARYD
YFTGTGVYWGQGTLVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSNTKVDKRVESKYGPPCPPCPAPEFLGGPSVFLFPPKPKDTL
MISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLG
SEQ ID NO: 79 IL-17 kappa LC
DIVMTQTPLSLSVTPGQPASISCRSSRSLVHSRGNTYLHWYLQKPGQSPQ
LLIYKVSNRFIGVPDRFSGSGSGTDFTLKISRVEAEDVGVYYCSQSTHLP
FTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
SEQ ID NO: 80 hIL-17 with C-terminal FlagHis tag
GITIPRNPGCPNSEDKNFPRTVMVNLNIHNRNTNTNPKRSSDYYNRSTS
PWNLHRNEDPERYPSVIWEAKCRHLGCINADGNVDYHMNSVPIQQEILV
LRREPPHCPNSFRLEKILVSVGCTCVTPIVHHVAAFIDYKDDDDKHVHH
HHHH
SEQ ID NO: 81 Hinge-Linker
ESSCDGGGGSGGGGSGGGGSGGGGS
SEQ ID NO: 82 Hinge-Linker
EPKSCDGGGGSGGGGSGGGGSGGGGS

1-14. (canceled)

15. A compound comprising a first polypeptide chain, a second polypeptide chain, and a third polypeptide chain, in which
a. the first polypeptide chain has the formula VL₁-Cβ, wherein
i. VL₁ is a first antibody light chain variable region, and
ii. Cβ is a T-Cell Receptor β constant domain (Cβ); and
b. the second polypeptide has the formula VL₂-CL, wherein
i. VL₂ is a second antibody light chain variable region, and
ii. CL is a light chain κ or λ constant domain; and
c. the third polypeptide chain has a formula selected from the group consisting of
(VH₁-Cα)-X1-(C_{H2}-C_{H3})-X2-(VH₂-C_{H1}),
(VH₂-C_{H1})-(H-C_{H2}-C_{H3})-X2-(V_{H1}-Cα),
(VH₁-Cα)-X2-(VH₂-C_{H1})-(H-C_{H2}-C_{H3}),
and
(VH₂-C_{H1})-X2-(VH₁-Cα)-X1-(C_{H2}-C_{H3}),
wherein
VH₁ is a first antibody heavy chain variable region,
Cα is a T-Cell Receptor α constant domain (Cα),
X₁ is a peptide linker or is absent,
C_{H2} C_{H3} are heavy chain constant domains 2 and 3,
H is a hinge region,
X₂ is a peptide linker or is absent,
VH₂ is a second antibody heavy chain variable region and
C_{H1} is heavy chain constant domain 1; and
in which the first and second polypeptide chains independently associate with the third polypeptide chain to form a first antigen binding site (VH₁-VL₁) and a second antigen binding site (VH₂-VL₂).

16. The compound of claim 15, wherein Cα is selected from the group consisting of SEQ ID NO:1, SEQ ID NO:2, and SEQ ID NO:7.

17. The compound of claim 15, wherein Cβ is selected from the group consisting of SEQ ID NO:10, SEQ ID NO:11, and SEQ ID NO:12.

18. The compound of claim 15, wherein C_{H2}-C_{H3} is SEQ ID NO:15; C_{H1} is SEQ ID NO:14; X₂ is SEQ ID NO:13; and X₁ is SEQ ID NO:5 or SEQ ID NO:6.

19. The compound of claim 15, wherein Cα is SEQ ID NO:2; Cβ is SEQ ID NO:11 or SEQ ID NO:12; C_{H2}-C_{H3} is SEQ ID NO:15; C_{H1} is SEQ ID NO:14; X₂ is SEQ ID NO:13; X₁ is SEQ ID NO:5 or SEQ ID NO:6.

20. The compound of claim 19 wherein Cβ is SEQ ID NO:11; X₁ is SEQ ID NO:6; and CL is SEQ ID NO:21.

21. The compound of claim 19 wherein Cβ is SEQ ID NO:12; X₁ is SEQ ID NO:6; and CL is SEQ ID NO:21.

22. The compound of claim 15, comprising two of each of the first, second, and third polypeptides, in which a first polypeptide chain and a second polypeptide chain independently associate with one of the third polypeptide chains to form a first antigen binding site and a second antigen binding site, and the other first polypeptide chain and the other second polypeptide chain associate with the other third polypeptide chain to form another first antigen binding site and another second antigen binding site.

23. The compound of claim 20, comprising two of each of the first, second, and third polypeptides, in which a first polypeptide chain and a second polypeptide chain independently associate with one of the third polypeptide chains to form a first antigen binding site and a second antigen binding site, and the other first polypeptide chain and the other second polypeptide chain associate with the other third polypeptide chain to form another first antigen binding site and another second antigen binding site.

24. The compound of claim 21, comprising two of each of the first, second, and third polypeptides, in which a first polypeptide chain and a second polypeptide chain independently associate with one of the third polypeptide chains to form a first antigen binding site and a second antigen binding site, and the other first polypeptide chain and the other second polypeptide chain associate with the other third polypeptide chain to form another first antigen binding site and another second antigen binding site.

25. A DNA molecule comprising a polynucleotide sequence encoding a polypeptide chain having a formula selected from the group consisting of
(VH₁-Cα)-X₂-(VH₂-C_{H1})-(C_{H2}-C_{H3}),
(VH₁-Cα)-X₁-(C_{H2}-C_{H3})-X₂-(VH₂-C_{H1}),
(VH₂-C_{H1})-(C_{H2}-C_{H3})-X₂-(VH₁-Cα),
and
(VH₂-C_{H1})-X₂-(VH₁-Cα)-X₁-(C_{H2}-C_{H3}),
in which
VH₁ is a first antibody heavy chain variable region,
Cα is a T-Cell Receptor α constant domain (Cα),
X₁ is a peptide linker or is absent,
C_{H2}-C_{H3} are heavy chain constant domains 2 and 3,
X₂ is a peptide linker or is absent,
VH₂ is a second antibody heavy chain variable region and
C_{H1} is a heavy chain constant domain 1.

26. A mammalian cell comprising DNA encoding three polypeptide chains,
a. wherein a first polypeptide chain has the formula VL₁-Cβ, wherein
VL₁ is a first antibody light chain variable region, and
Cβ is a T-Cell Receptor β constant domain (Cβ);
b. the second polypeptide chain has the formula VL₂-C_{L}, wherein
V_{L2} is a second antibody light chain variable region, and
CL is a light chain κ or λ constant domain; and
c. the third polypeptide chain has a formula selected from the group consisting of
(VH₁-Cα)-X₁-(C_{H2}-C_{H3})-X₂-(VH₂-C_{H1}),
(VH₂-C_{H1})-(H-C_{H2}-C_{H3})-X₂-(VH₁-Cα),
(VH₁-Cα)-X₂-(VH₂-C_{H1})-(H-C_{H2}-C_{H3}),
and
(VH₂-C_{H1})-X₂-(VH₁-Cα)-X₁-(C_{H2}-C_{H3}),
wherein
VH₁ is a first antibody heavy chain variable region,
Cα is a T-Cell Receptor α constant domain (Cα),
X₁ is a peptide linker or is absent,
C_{H2}-C_{H3} are heavy chain constant domains 2 and 3,
H is a hinge region between C_{H1} and C_{H2},
X₂ is a peptide linker or is absent,
VH₂ is a second antibody heavy chain variable region and
C_{H1} is a heavy chain constant domain 1.

27. A process for producing a compound comprising a first polypeptide chain, a second polypeptide chain, and a third polypeptide chain, in which
a. the first polypeptide chain has the formula VL₁-Cβ, wherein
i. VL₁ is a first antibody light chain variable region, and
ii. Cβ is a T-Cell Receptor β constant domain (Cβ); and
b. the second polypeptide has the formula VL₂-CL, wherein
i. VL₂ is a second antibody light chain variable region, and
ii. CL is a light chain κ or λ constant domain; and
c. the third polypeptide chain has a formula selected from the group consisting of
(VH₁-Cα)-X1-(C_{H2}-C_{H3})-X2-(VH₂-C_{H1}),
(VH₂-C_{H1})-(H-C_{H2}-C_{H3})-X2-(V_{H1}-Cα),
(VH₁-Cα)-X2-(VH₂-C_{H1})-(H-C_{H2}-C_{H3}),
and
(VH₂-C_{H1})-X2-(VH₁-Cα)-X1-(C_{H2}-C_{H3}),
wherein
VH₁ is a first antibody heavy chain variable region,
Cα is a T-Cell Receptor α constant domain (Cα),
X₁ is a peptide linker or is absent,
C_{H2}-C_{H3} are heavy chain constant domains 2 and 3,
H is a hinge region,
X₂ is a peptide linker or is absent,
VH₂ is a second antibody heavy chain variable region and
C_{H1} is heavy chain constant domain 1;
in which the first and second polypeptide chains independently associate with the third polypeptide chain to form a first antigen binding site (VH₁-VL₁) and a second antigen binding site (VH₂-VL₂),
comprising cultivating the cell of claim 26 under conditions such that the compound is expressed and recovering the compound.

28. A compound produced by the process of claim 27.
